SEQUENCE LISTING (8 sequences)

SEQ ID NO: 1 - DNA, 33 bp, Artificial Sequence
  PCR primers
ggaattccaaccatgggagtgaggcacccgccc

SEQ ID NO: 2 - DNA, 33 bp, Artificial Sequence
  PCR primers
gctctagactaacaccagctgacgagaaactgc

SEQ ID NO: 3 - DNA, 276 bp, Artificial Sequence
  A DNA cassette for the production of recombinant GILT-tagged GAA ZC-701
gaattcacaccaatgggaatcccaatggggaagtcgatgctggtgcttctcaccttcttggccttcgcctcgtgctgcattgctgctctgtgcggcggggagctggtggacaccctccagttcgtctgtggggaccgcggcttctacttcagcaggcccgcaagccgtgtgagccgtcgcagccgtggcatcgttgaggagtgctgtttccgcagctgtgacctggccctcctggagacgtactgtgctacccccgccaagtccgagggcgcgccg

SEQ ID NO: 4 - protein, 971 aa, Artificial Sequence
  Cassette 701 was digested with EcoRI and XbaI, blunted by treatment with Klenow DNA polymerase
MGIPMGKSMLVLLTFLAFASCCIAALCGGELVDTLQFVCGDRGFYFSRPASRVSRRSRGIVEECCFRSCDLALLETYCATPAKSEGAPAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPRVHSRAPSPLYSVEFSEEPFGVIVHRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPIEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 5 - DNA, 2040 bp, Artificial Sequence
  His-Biotin-Ci-Mpr Domains 10-13
atgggaatcccaatggggaagtcgatgctggtgcttctcaccttcttggccttcgcctcgtgctgcattgctgctggcgcgccgaccggtcaccatcaccatcaccacgcgccgggcctgaacgacatcttcgaggcccagaagatcgagtggcacgaacctttcgatctgactgaatgttcattcaaagatggggctggcaactccttcgacctctcgtccctgtcaaggtacagtgacaactgggaagccatcactgggacgggggacccggagcactacctcatcaatgtctgcaagtctctggccccgcaggctggcactgagccgtgccctccagaagcagccgcgtgtctgctgggtggctccaagcccgtgaacctcggcagggtaagggacggacctcagtggagagatggcataattgtcctgaaatacgttgatggcgacttatgtccagatgggattcggaaaaagtcaaccaccatccgattcacctgcagcgagagccaagtgaactccaggcccatgttcatcagcgccgtggaggactgtgagtacacctttgcctggcccacagccacagcctgtcccatgaagagcaacgagcatgatgactgccaggtcaccaacccaagcacaggacacctgtttgatctgagctccttaagtggcagggcgggattcacagctgcttacagcgagaaggggttggtttacatgagcatctgtggggagaatgaaaactgccctcctggcgtgggggcctgctttggacagaccaggattagcgtgggcaaggccaacaagaggctgagatacgtggaccaggtcctgcagctggtgtacaaggatgggtccccttgtccctccaaatccggcctgagctataagagtgtgatcagtttcgtgtgcaggcctgaggccgggccaaccaataggcccatgctcatctccctggacaagcagacatgcactctcttcttctcctggcacacgccgctggcctgcgagcaagcgaccgaatgttccgtgaggaatggaagctctattgttgacttgtctccccttattcatcgcactggtggttatgaggcttatgatgagagtgaggatgatgcctccgataccaaccctgatttctacatcaatatttgtcagccactaaatcccatgcacggagtgccctgtcctgccggagccgctgtgtgcaaagttcctattgatggtccccccatagatatcggccgggtagcaggaccaccaatactcaatccaatagcaaatgagatttacttgaattttgaaagcagtactccttgcttagcggacaagcatttcaactacacctcgctcatcgcgtttcactgtaagagaggtgtgagcatgggaacgcctaagctgttaaggaccagcgagtgcgactttgtgttcgaatgggagactcctgtcgtctgtcctgatgaagtgaggatggatggctgtaccctgacagatgagcagctcctctacagcttcaacttgtccagcctttccacgagcacctttaaggtgactcgcgactcgcgcacctacagcgttggggtgtgcacctttgcagtcgggccagaacaaggaggctgtaaggacggaggagtctgtctgctctcaggcaccaagggggcatcctttggacggctgcaatcaatgaaactggattacaggcaccaggatgaagcggtcgttttaagttacgtgaatggtgatcgttgccctccagaaaccgatgacggcgtcccctgtgtcttccccttcatattcaatgggaagagctacgaggagtgcatcatagagagcagggcgaagctgtggtgtagcacaactgcggactacgacagagaccacgagtggggcttctgcagacactcaaacagctaccggacatccagcatcatatttaagtgtgatgaagatgaggacattgggaggccacaagtcttcagtgaagtgcgtgggtgtgatgtgacatttgagtggaaaacaaaagttgtctgcccttga

SEQ ID NO: 6 - protein, 679 aa, Artificial Sequence
  His-Biotin-Ci-Mpr Domains 10-13 protein sequences
MGIPMGKSMLVLLTFLAFASCCIAAGAPTGHHHHHHAPGLNDIFEAQKIEWHEPFDLTECSFKDGAGNSFDLSSLSRYSDNWEAITGTGDPEHYLINVCKSLAPQAGTEPCPPEAAACLLGGSKPVNLGRVRDGPQWRDGIIVLKYVDGDLCPDGIRKKSTTIRFTCSESQVNSRPMFISAVEDCEYTFAWPTATACPMKSNEHDDCQVTNPSTGHLFDLSSLSGRAGFTAAYSEKGLVYMSICGENENCPPGVGACFGQTRISVGKANKRLRYVDQVLQLVYKDGSPCPSKSGLSYKSVISFVCRPEAGPTNRPMLISLDKQTCTLFFSWHTPLACEQATECSVRNGSSIVDLSPLIHRTGGYEAYDESEDDASDTNPDFYINICQPLNPMHGVPCPAGAAVCKVPIDGPPIDIGRVAGPPILNPIANEIYLNFESSTPCLADKHFNYTSLIAFHCKRGVSMGTPKLLRTSECDFVFEWETPVVCPDEVRMDGCTLTDEQLLYSFNLSSLSTSTFKVTRDSRTYSVGVCTFAVGPEQGGCKDGGVCLLSGTKGASFGRLQSMKLDYRHQDEAVVLSYVNGDRCPPETDDGVPCVFPFIFNGKSYEECIIESRAKLWCSTTADYDRDHEWGFCRHSNSYRTSSIIFKCDEDEDIGRPQVFSEVRGCDVTFEWKTKVVCP

SEQ ID NO: 7 - DNA, 2040 bp, Artificial Sequence
  His-Biotin-Ci-Mpr Domains 10-13 I1572T.  The underlined sequence change results in the point mutation I1572T and a silent mutation S1573 that creates a diagnostic SpeI site.
atgggaatcccaatggggaagtcgatgctggtgcttctcaccttcttggccttcgcctcgtgctgcattgctgctggcgcgccgaccggtcaccatcaccatcaccacgcgccgggcctgaacgacatcttcgaggcccagaagatcgagtggcacgaacctttcgatctgactgaatgttcattcaaagatggggctggcaactccttcgacctctcgtccctgtcaaggtacagtgacaactgggaagccatcactgggacgggggacccggagcactacctcatcaatgtctgcaagtctctggccccgcaggctggcactgagccgtgccctccagaagcagccgcgtgtctgctgggtggctccaagcccgtgaacctcggcagggtaagggacggacctcagtggagagatggcataattgtcctgaaatacgttgatggcgacttatgtccagatgggattcggaaaaagtcaaccaccatccgattcacctgcagcgagagccaagtgaactccaggcccatgttcatcagcgccgtggaggactgtgagtacacctttgcctggcccacagccacagcctgtcccatgaagagcaacgagcatgatgactgccaggtcaccaacccaagcacaggacacctgtttgatctgagctccttaagtggcagggcgggattcacagctgcttacagcgagaaggggttggtttacatgagcatctgtggggagaatgaaaactgccctcctggcgtgggggcctgctttggacagaccaggactagtgtgggcaaggccaacaagaggctgagatacgtggaccaggtcctgcagctggtgtacaaggatgggtccccttgtccctccaaatccggcctgagctataagagtgtgatcagtttcgtgtgcaggcctgaggccgggccaaccaataggcccatgctcatctccctggacaagcagacatgcactctcttcttctcctggcacacgccgctggcctgcgagcaagcgaccgaatgttccgtgaggaatggaagctctattgttgacttgtctccccttattcatcgcactggtggttatgaggcttatgatgagagtgaggatgatgcctccgataccaaccctgatttctacatcaatatttgtcagccactaaatcccatgcacggagtgccctgtcctgccggagccgctgtgtgcaaagttcctattgatggtccccccatagatatcggccgggtagcaggaccaccaatactcaatccaatagcaaatgagatttacttgaattttgaaagcagtactccttgcttagcggacaagcatttcaactacacctcgctcatcgcgtttcactgtaagagaggtgtgagcatgggaacgcctaagctgttaaggaccagcgagtgcgactttgtgttcgaatgggagactcctgtcgtctgtcctgatgaagtgaggatggatggctgtaccctgacagatgagcagctcctctacagcttcaacttgtccagcctttccacgagcacctttaaggtgactcgcgactcgcgcacctacagcgttggggtgtgcacctttgcagtcgggccagaacaaggaggctgtaaggacggaggagtctgtctgctctcaggcaccaagggggcatcctttggacggctgcaatcaatgaaactggattacaggcaccaggatgaagcggtcgttttaagttacgtgaatggtgatcgttgccctccagaaaccgatgacggcgtcccctgtgtcttccccttcatattcaatgggaagagctacgaggagtgcatcatagagagcagggcgaagctgtggtgtagcacaactgcggactacgacagagaccacgagtggggcttctgcagacactcaaacagctaccggacatccagcatcatatttaagtgtgatgaagatgaggacattgggaggccacaagtcttcagtgaagtgcgtgggtgtgatgtgacatttgagtggaaaacaaaagttgtctgcccttga

SEQ ID NO: 8 - protein, 679 aa, Artificial Sequence
  His-Biotin-Ci-Mpr Domains 10-13 I1572T protein sequence. The I1572T mutation is underlined.
MGIPMGKSMLVLLTFLAFASCCIAAGAPTGHHHHHHAPGLNDIFEAQKIEWHEPFDLTECSFKDGAGNSFDLSSLSRYSDNWEAITGTGDPEHYLINVCKSLAPQAGTEPCPPEAAACLLGGSKPVNLGRVRDGPQWRDGIIVLKYVDGDLCPDGIRKKSTTIRFTCSESQVNSRPMFISAVEDCEYTFAWPTATACPMKSNEHDDCQVTNPSTGHLFDLSSLSGRAGFTAAYSEKGLVYMSICGENENCPPGVGACFGQTRTSVGKANKRLRYVDQVLQLVYKDGSPCPSKSGLSYKSVISFVCRPEAGPTNRPMLISLDKQTCTLFFSWHTPLACEQATECSVRNGSSIVDLSPLIHRTGGYEAYDESEDDASDTNPDFYINICQPLNPMHGVPCPAGAAVCKVPIDGPPIDIGRVAGPPILNPIANEIYLNFESSTPCLADKHFNYTSLIAFHCKRGVSMGTPKLLRTSECDFVFEWETPVVCPDEVRMDGCTLTDEQLLYSFNLSSLSTSTFKVTRDSRTYSVGVCTFAVGPEQGGCKDGGVCLLSGTKGASFGRLQSMKLDYRHQDEAVVLSYVNGDRCPPETDDGVPCVFPFIFNGKSYEECIIESRAKLWCSTTADYDRDHEWGFCRHSNSYRTSSIIFKCDEDEDIGRPQVFSEVRGCDVTFEWKTKVVCP

(19) United States
(12) Patent Application Publication
(10) Pub. No.: US 2009/0117091 A1
(43) Pub. Date: May 7, 2009

(54) Methods for treating pompe disease

(76) Inventors: Jonathan LeBowitz, Whitefish Bay, WI (US); John Maga, Whitefish Bay, WI (US)

Correspondence Address: CHOATE, HALL & STEWART LLP, TWO INTERNATIONAL PLACE, BOSTON, MA, 02110, US

(21) Appl. No.: 11/985,360

(22) Filed: Nov. 13, 2007

Related U.S. Application Data
(60) Provisional application No. 60/858,514, filed on Nov. 13, 2006.
(60) Provisional application No. 60/879,255, filed on Jan. 5, 2007.
(60) Provisional application No. 60/900,187, filed on Feb. 7, 2007.

(51) Int. Cl. A61K 38/47

(52) U.S. Cl. 424/94.61

(57) ABSTRACT

The present invention provides methods for treating Pompe disease in a subject by administering to the subject a therapeutically effective amount of a fusion protein which includes human acid alpha-glucosidase (GAA), or a fragment thereof, and a lysosomal targeting domain. The lysosomal targeting domain binds the human cation-independent mannose-6-phosphate receptor in a mannose-6-phosphate-independent manner.

### REFERENCE TO RELATED APPLICATIONS

This application the benefit of U.S. Provisional Patent Application No. 60/900,187, filed Feb. 7, 2007; U.S. Provisional Patent Application No. 60/879,255, filed Jan. 5, 2007; U.S. Provisional Patent Application No. 60/858,514, filed Nov. 13, 2006, the contents of each of which are hereby incorporated by reference in their entireties. This application also relates to U.S. patent application Ser. No. 11/057,058, filed Feb. 10, 2005, the contents of which are hereby incorporated by reference in their entireties.

### FIELD OF THE INVENTION

The invention relates to methods and compositions for treating Pompe disease. In particular, the invention relates to therapeutic methods for treating Pompe disease by targeting acid alpha-glucosidase to the lysosome in a mannose-6-phosphate-independent manner.

### BACKGROUND

Pompe disease is an autosomal recessive genetic disorder caused by a deficiency or dysfunction of the lysosomal hydrolase acid alpha-glucosidase (GAA), a glycogen-degrading lysosomal enzyme. Deficiency of GAA results in lysosomal glycogen accumulation in many tissues in Pompe patients, with cardiac and skeletal muscle tissues most seriously affected. The combined incidence of all forms of Pompe disease is estimated to be 1:40,000, and the disease affects all groups without an ethnic predilection. It is estimated that approximately one third of those with Pompe disease have the rapidly progressive, fatal infantile-onset form, while the majority of patients present with the more slowly progressive, juvenile or late-onset forms.

Drug treatment strategies, dietary manipulations, and bone marrow transplantation have been employed as means for treatment of Pompe disease, without significant success. In recent years, enzyme replacement therapy (ERT) has provided new hope for Pompe patients. For example, Myozyme®, a recombinant GAA protein drug, received approval for use in patients with Pompe disease in 2006 in both the U.S. and Europe. Myozyme® depends on mannose-6-phosphates (M6P) on the surface of the GAA protein for delivery to lysosomes.

### SUMMARY OF THE INVENTION

The present invention provides new and improved methods for treating Pompe disease. Specifically, the present invention provides methods and compositions for targeting acid alpha-glucosidase (GAA) to lysosomes in a mannose-6-phosphate independent manner. As a result, the methods of the present invention are simpler, more efficient, more potent, and more cost-effective. The present invention thus significantly advances the progress of enzyme replacement therapy for Pompe disease.

In one aspect, the present invention provides a method for treating Pompe disease in a subject by administering to the subject a therapeutically effective amount of a fusion protein. The fusion protein includes human acid alpha-glucosidase (GAA) (or a fragment of human GAA) and a lysosomal targeting domain. The lysosomal targeting domain binds the human cation-independent mannose-6-phosphate receptor in a mannose-6-phosphate-independent manner.

In one embodiment, the lysosomal targeting domain includes mature human insulin-like growth factor II (IGF-II), or a fragment or sequence variant of mature human IGF-II. In one embodiment, the lysosomal targeting domain includes amino acids 8-67 of mature human IGF-II. Preferably, the lysosomal targeting domain includes amino acids 1 and 8-67 of mature human IGF-II (i. e., Δ2-7 of mature human GAA). In another embodiment, the fusion protein includes amino acids 70-952 of human GAA.

In one embodiment, the fusion protein suitable for the present invention has a reduced mannose-6-phosphate (M6P) level on the surface of the protein compared to wild-type human GAA. In yet another embodiment, the fusion protein suitable for the present invention has no functional M6P level on the surface of the protein.

In another embodiment, the therapeutically effective amount is in the range of about 2.5-20 milligram per kilogram of body weight of the subject (mg/kg).

In one embodiment, the fusion protein is administered intravenously. In other embodiments, the fusion protein is administered bimonthly, monthly, triweekly, biweekly, weekly, daily, or at variable intervals. As used herein, the term “bimonthly” means administration once per two months (i.e., once every two months); the term “monthly” means administration once per month; the term “triweekly” means administration once per three weeks (i.e., once every three weeks); the term “biweekly” means administration once per two weeks (i.e., once every two weeks); the term “weekly” means administration once per week; and the term “daily” means administration once per day.

In further embodiments, the fusion protein is administered in conjunction with an immunosuppressant. The immunosuppressant can be administered prior to any administration of the fusion protein. In some embodiments, the method for treating Pompe disease further includes the additional step of tolerizing the subject.

Another aspect of the invention provides a method for treating Pompe disease in a subject by administering to the subject a therapeutically effective amount of a fusion protein. The fusion protein includes amino acids 1 and 8-67 of mature human insulin-like growth factor II (IGF-II) (i.e., Δ2-7 of mature human GAA) and amino acids 70-952 of human acid alpha-glucosidase (GAA). In a preferred embodiment, the fusion protein includes the spacer sequence Gly-Ala-Pro between the amino acids of human GAA and the amino acids of mature human IGF-II.

In one embodiment, the fusion protein suitable for this aspect of the invention has a reduced mannose-6-phosphate (M6P) level on the surface of the protein compared to wild-type human GAA. In yet another embodiment, the fusion protein suitable for this aspect of the invention has no functional M6P level on the surface of the protein.

A further aspect of the invention provides a method for reducing glycogen levels in vivo by administering to a subject suffering from Pompe disease an effective amount of a fusion protein. The fusion protein includes human acid alpha-glucosidase (GAA) (or a fragment of human GAA) and a lysosomal targeting domain. The lysosomal targeting domain binds the human cation-independent mannose-6-phosphate receptor in a mannose-6-phosphate-independent manner.

In one embodiment, the lysosomal targeting domain includes mature human insulin-like growth factor II (IGF-II), or a fragment or sequence variant of mature human IGF-II. In one embodiment, the lysosomal targeting domain includes amino acids 8-67 of mature human IGF-II. Preferably, the lysosomal targeting domain includes amino acids 1 and 8-67 of mature human IGF-II (i.e., Δ2-7 of mature human GAA). In another preferred embodiment, the fusion protein includes amino acids 70-952 of human GAA.

In one embodiment, the fusion protein suitable for this aspect of the invention has a reduced mannose-6-phosphate (M6P) level on the surface of the protein compared to wild-type human GAA. In yet another embodiment, the fusion protein suitable for this aspect of the invention has no functional M6P level on the surface of the protein.

In another embodiment, the effective amount is in the range of about 2.5-20 milligram per kilogram of body weight of the subject (mg/kg).

In some embodiments, the fusion protein is administered intravenously. In other embodiments, the fusion protein is administered bimonthly, monthly, triweekly, biweekly, weekly, daily, or at variable intervals.

In another aspect, the invention provides a method for reducing glycogen levels in a mammalian lysosome by targeting to the lysosome an effective amount of a fusion protein. The fusion protein includes human acid alpha-glucosidase (GAA) (or a fragment of human GAA) and a lysosomal targeting domain. The lysosomal targeting domain binds the human cation-independent mannose-6-phosphate receptor in a mannose-6-phosphate-independent manner.

In one embodiment, the lysosomal targeting domain includes human insulin-like growth factor II (IGF-II), or a fragment or sequence variant of human IGF-II. In one embodiment, the lysosomal targeting domain includes amino acids 8-67 of mature human IGF-II. Preferably, the lysosomal targeting domain includes amino acids 1 and 8-67 of mature human IGF-II (i.e., Δ2-7 of mature human GAA). In another preferred embodiment, the fusion protein includes amino acids 70-952 of human GAA.

In another aspect, the invention provides a method for reducing glycogen levels in a muscle tissue of a subject suffering from Pompe disease by delivering to the muscle tissue a therapeutically effective amount of a fusion protein. The fusion protein includes human acid alpha-glucosidase (GAA) (or a fragment of human GAA) and a lysosomal targeting domain. The lysosomal targeting domain binds the human cation-independent mannose-6-phosphate receptor in a mannose-6-phosphate-independent manner. In one embodiment, the muscle tissue is skeletal muscle.

Another aspect of the invention provides a method for treating cardiomyopathy associated with Pompe disease in a subject by administering to the subject a therapeutically effective amount of a fusion protein. The fusion protein includes human acid alpha-glucosidase (GAA) (or a fragment of human GAA) and a lysosomal targeting domain. The lysosomal targeting domain binds the human cation-independent mannose-6-phosphate receptor in a mannose-6-phosphate-independent manner.

In yet another aspect, the invention provides a method for treating myopathy associated with Pompe disease in a subject by administering to the subject a therapeutically effective amount of a fusion protein. The fusion protein includes human acid alpha-glucosidase (GAA) (or a fragment of human GAA) and a lysosomal targeting domain. The lysosomal targeting domain binds the human cation-independent mannose-6-phosphate receptor in a mannose-6-phosphate-independent manner.

Another aspect of the invention provides a method for increasing acid alpha-glucosidase activity in a subject suffering from Pompe disease by administering to the subject a fusion protein which includes human acid alpha-glucosidase (GAA) (or a fragment of human GAA) and a lysosomal targeting domain. The lysosomal targeting domain binds the human cation-independent mannose-6-phosphate receptor in a mannose-6-phosphate-independent manner.

A further aspect of the invention provides a pharmaceutical composition suitable for the treatment of Pompe disease. The pharmaceutical composition includes a therapeutically effective amount of a fusion protein which includes human acid alpha-glucosidase (GAA) (or a fragment of human GAA) and a lysosomal targeting domain. The lysosomal targeting domain binds the human cation-independent mannose-6-phosphate receptor in a mannose-6-phosphate-independent manner.

In one embodiment, the lysosomal targeting domain includes mature human insulin-like growth factor II (IGF-II), or a fragment or sequence variant of mature human IGF-II. In one embodiment, the lysosomal targeting domain includes amino acids 8-67 of mature human IGF-II. Preferably, the lysosomal targeting domain includes amino acids 1 and 8-67 of mature human IGF-II (i.e., Δ2-7 of mature human GAA). In another preferred embodiment, the fusion protein includes amino acids 70-952 of human GAA.

In another embodiment, the fusion protein includes amino acids 70-952 of human GAA and amino acids 1 and 8-67 of mature human IGF-II (i.e., Δ2-7 of mature human GAA). In a further embodiment, the fusion protein further includes the spacer sequence Gly-Ala-Pro between the fragment of mature human IGF-II (amino acids 1 and 8-67) and the fragment of human GAA (amino acids 70-952).

In one embodiment, the fusion protein suitable for this aspect of the invention has reduced mannose-6-phosphate (M6P) level on the surface of the protein compared to wild-type human GAA. In yet another embodiment, the fusion protein suitable for this aspect of the invention has no functional M6P level on the surface of the protein.

In yet another embodiment, the pharmaceutical composition includes a pharmaceutical carrier.

As used in this application, “human acid alpha-glucosidase (GAA)” refers to precursor wild-type form of human GAA or a functional variant that is capable of reducing glycogen levels in mammalian lysosomes or that can rescue or ameliorate one or more Pompe disease symptoms.

As used in this application, the terms “about” and “approximately” are used as equivalents. Any numerals used in this application with or without about/approximately are meant to cover any normal fluctuations appreciated by one of ordinary skill in the relevant art.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWINGS

The drawings are for illustration purposes only, not for limitation.

FIG. 1 shows a schematic representation of GILT-tagged GAA ZC-701.

FIGS. 2A-C show SDS-PAGE and Western blots of wild-type, untagged GAA and GILT-tagged GAA ZC-701. FIG. 2A shows SDS-PAGE followed by silver staining. FIG. 2B shows a Western blot using anti-GAA antibody. FIG. 2C shows a Western blot using anti-IGF-II antibody.

FIG. 3A shows schematic representations of p1288 and p1355, two biotinylated and His-tagged recombinant proteins containing wild-type CI-MPR domains 10-13 and a point mutant variant, respectively.

FIG. 3B depicts expression of 1288 and 1355 by silver stain.

FIGS. 4A-B depict exemplary results of Biacore® analysis of GILT-tagged GAA ZC-701 interactions with CI-MPR. FIG. 4A depicts exemplary binding curves for IGF-II. FIG. 4B depicts exemplary binding curves for GILT-tagged GAA ZC-701.

FIG. 5 depicts exemplary results of tag-dependent uptake of GILT-tagged GAA ZC-701 into rat L6 myoblasts.

FIG. 6 depicts exemplary saturation curves for uptake of purified GILT-tagged GAA ZC-701 and wild-type untagged GAA into rat L6 Myoblasts.

FIG. 7 depicts exemplary results reflecting the half-life of GILT-tagged GAA ZC-701 and wild-type, untagged GAA (ZC-635) in rat L6 myoblasts.

FIGS. 8A-B are exemplary Western blots showing proteolytic processing of GILT-tagged GAA ZC-701 after uptake into rat L6 myoblasts. FIG. 8A is an exemplary Western blot showing loss of the GILT tag after uptake. FIG. 8B is an exemplary Western blot showing processing of wild-type and GILT-tagged GAA into various peptide species after uptake.

FIG. 9 depicts exemplary results reflecting the serum half-life in wild-type 129 mice of GILT-tagged GAA ZC-701 produced in three different tissue culture media. The red line corresponds to PF-CHO media, t½=43 min; the orange line corresponds to CDM4 media, t½=38 min; and the green line corresponds to CD17 media, t½=52 min.

FIGS. 10A-D depict exemplary decay curves in various tissues of Pompe mice for wild-type, untagged GAA (ZC-635); GILT-tagged GAA ZC-701; and GILT-tagged GAA ZC-1026. FIG. 10A depicts exemplary decay curves in quadriceps tissue. FIG. 10B depicts exemplary decay curves in heart tissue. FIG. 10C depicts exemplary decay curves in diaphragm tissue. FIG. 10D depicts exemplary decay curves in liver tissue.

FIG. 11 depicts the co-localization of GILT-tagged GAA and a lysosomal marker, LAMPI.

FIG. 12 depicts exemplary results demonstrating clearance of glycogen in heart tissue samples taken from Pompe mice treated with a single injection of either GILT-tagged GAA protein, ZC-701, or an untagged GAA.

FIGS. 13A-H are exemplary graphs showing glycogen clearance in various muscle tissues of Pompe mice after injections of wild-type, untagged GAA or GILT-tagged GAA ZC-701.

FIG. 14 shows a detailed flowchart of clinical study procedures.

### DETAILED DESCRIPTION OF THE INVENTION

The present invention provides methods and compositions for treating Pompe disease based on the glycosylation-independent lysosomal targeting technology (GILT). In particular, the present invention provides methods and compositions for treating Pompe disease by targeting acid alpha-glucosidase to the lysosome in a mannose-6-phosphate-independent manner.

Various aspects of the invention are described in detail in the following sections. The use of sections is not meant to limit the invention. Each section can apply to any aspect of the invention. In this application, the use of “or” means “and/or” unless stated otherwise.

### Pompe Disease

Pompe disease is a rare genetic disorder caused by a deficiency in the enzyme acid alpha-glucosidase (GAA), which is needed to break down glycogen, a stored form of sugar used for energy. Pompe disease is also known as glycogen storage disease type II, GSD II, type II glycogen storage disease, glycogenosis type II, acid maltase deficiency, alpha-1,4-glucosidase deficiency, cardiomegalia glycogenic diffusa, and cardiac form of generalized glycogenosis. The build-up of glycogen causes progressive muscle weakness (myopathy) throughout the body and affects various body tissues, particularly in the heart, skeletal muscles, liver, respiratory and nervous system.

The presenting clinical manifestations of Pompe disease can vary widely depending on the age of disease onset and residual GAA activity. Residual GAA activity correlates with both the amount and tissue distribution of glycogen accumulation as well as the severity of the disease. Infantile-onset Pompe disease (less than 1% of normal GAA activity) is the most severe form and is characterized by hypotonia, generalized muscle weakness, and hypertrophic cardiomyopathy, and massive glycogen accumulation in cardiac and other muscle tissues. Death usually occurs within one year of birth due to cardiorespiratory failure. Hirschhorn et al. (2001) “Glycogen Storage Disease Type II: Acid Alpha-glucosidase (Acid Maltase) Deficiency,” in Scriver et al., eds., The Metabolic and Molecular Basis of Inherited Disease, 8th Ed., New York: McGraw-Hill, 3389-3420. Juvenile-onset (1-10% of normal GAA activity) and adult-onset (10-40% of normal GAA activity) Pompe disease are more clinically heterogeneous, with greater variation in age of onset, clinical presentation, and disease progression. Juvenile- and adult-onset Pompe disease are generally characterized by lack of severe cardiac involvement, later age of onset, and slower disease progression, but eventual respiratory or limb muscle involvement results in significant morbidity and mortality. While life expectancy can vary, death generally occurs due to respiratory failure. Hirschhorn et al. (2001) “Glycogen Storage Disease Type II: Acid Alpha-glucosidase (Acid Maltase) Deficiency,” in Scriver et al., eds., The Metabolic and Molecular Basis of Inherited Disease, 8th Ed., New York: McGraw-Hill, 3389-3420.

### Enzyme Replacement Therapy

Enzyme replacement therapy (ERT) is a therapeutic strategy to correct an enzyme deficiency by infusing the missing enzyme into the bloodstream. As the blood perfuses patient tissues, enzyme is taken up by cells and transported to the lysosome, where the enzyme acts to eliminate material that has accumulated in the lysosomes due to the enzyme deficiency. For lysosomal enzyme replacement therapy to be effective, the therapeutic enzyme must be delivered to lysosomes in the appropriate cells in tissues where the storage defect is manifest. Conventional lysosomal enzyme replacement therapeutics are delivered using carbohydrates naturally attached to the protein to engage specific receptors on the surface of the target cells. One receptor, the cation-independent M6P receptor (CI-MPR), is particularly useful for targeting replacement lysosomal enzymes because the CI-MPR is present on the surface of most cell types.

The terms “cation-independent mannose-6-phosphate receptor (CI-MPR)”, “M6P/IGF-II receptor,” and “CI-MPR/IGF-II receptor” are used interchangeably herein, referring to the cellular receptor which binds both M6P and IGF-II.

### Glycosylation Independent Lysosomal Targeting

The present invention developed a Glycosylation Independent Lysosomal Targeting (GILT) technology to target therapeutic enzymes to the lysosome. Specifically, the present invention uses a peptide tag instead of M6P to engage the CI-MPR for lysosomal targeting. Typically, a GILT tag is a protein, peptide, or other moiety that binds the CI-MPR in a mannose-6-phosphate-independent manner. Advantageously, this technology mimics the normal biological mechanism for uptake of lysosomal enzymes, yet does so in a manner independent of mannose-6-phosphate.

A preferred GILT tag is derived from human insulin-like growth factor II (IGF-II). Human IGF-II is a high affinity ligand for the CI-MPR, which is also referred to as IGF-II receptor. Binding of GILT-tagged therapeutic enzymes to the M6P/IGF-II receptor targets the protein to the lysosome via the endocytic pathway. This method has numerous advantages over methods involving glycosylation including simplicity and cost effectiveness, because once the protein is isolated, no further modifications need be made.

Detailed description of the GILT technology and GILT tag can be found in U.S. Publication Nos. 20030082176, 20040006008, 20040005309, and 20050281805, the teachings of all of which are hereby incorporated by references in their entireties.

### GILT-tagged GAA

By fusing a cassette encoding an appropriate GILT tag to a GAA-encoding sequence, the present invention provides a GILT-tagged GAA that can bind the CI-MPR with high affinity, independent of M6P content on the protein. In addition, the present invention provides a GAA preparation in which every enzyme molecule possesses a high affinity ligand for the CI-MPR. As described in the Example section, the GILT-tagged GAA has a high affinity for the CI-MPR by Biacore® analysis and is therapeutically more effective in vivo than conventional lysosomal enzyme replacement therapeutics.

The superior potency of GILT-tagged GAA provides a number of clinical benefits. The increased potency will simply result in a more favorable clinical prognosis at similar or lower doses. The GILT-tagged GAA can be delivered more efficiently to multiple tissues affected by the disease. For example, the GILT-tagged GAA can have increased delivery to skeletal muscles, in particular, at lower dosages. Increased potency may also permit a dose low enough to minimize adverse events that patients often suffer and to mitigate production of antibodies against the drug in patients. The increased potency may also permit a treatment regimen with increased intervals between infusions.

In a preferred embodiment, the GILT-tagged GAA includes a human GAA, or a fragment or sequence variant thereof which retains the ability to cleave α1-4 linkages in linear oligosaccharides, and a lysosomal targeting domain that binds the human CI-MPR in a mannose-6-phosphate-independent manner. A suitable lysosomal targeting domain includes mature human IGF-II, or a fragment or sequence variant thereof.

IGF-II is preferably targeted specifically to the CI-MPR. Particularly useful are mutations in the IGF-II polypeptide that result in a protein that binds the CI-MPR with high affinity while no longer binding the other IGF-II receptors with appreciable affinity. IGF-II can also be modified to minimize binding to serum IGF-binding proteins (Baxter (2000) Am. J. Physiol Endocrinol Metab. 278(6):967-76) to avoid sequestration of IGF-II/GILT constructs. A number of studies have localized residues in IGF-II necessary for binding to IGF-binding proteins. Constructs with mutations at these residues can be screened for retention of high affinity binding to the M6P/IGF-II receptor and for reduced affinity for IGF-binding proteins. For example, replacing Phe 26 of IGF-II with Ser is reported to reduce affinity of IGF-II for IGFBP-1 and -6 with no effect on binding to the M6P/IGF-II receptor (Bach et al. (1993) J. Biol. Chem. 268(13):9246-54). Other substitutions, such as Lys for Glu 9, can also be advantageous. The analogous mutations, separately or in combination, in a region of IGF-I that is highly conserved with IGF-II result in large decreases in IGF-BP binding (Magee et al. (1999) Biochemistry 38(48): 15863-70).

An alternate approach is to identify minimal regions of IGF-II that can bind with high affinity to the M6P/IGF-II receptor. The residues that have been implicated in IGF-II binding to the M6P/IGF-II receptor mostly cluster on one face of IGF-II (Terasawa et al. (1994) EMBO J. 13(23):5590-7). Although IGF-II tertiary structure is normally maintained by three intramolecular disulfide bonds, a peptide incorporating the amino acid sequence on the M6P/IGF-II receptor binding surface of IGF-II can be designed to fold properly and have binding activity. Such a minimal binding peptide is a highly preferred lysosomal targeting domain. For example, a preferred lysosomal targeting domain is amino acids 8-67 of human IGF-II. Designed peptides, based on the region around amino acids 48-55, which bind to the M6P/IGF-II receptor, are also desirable lysosomal targeting domains. Alternatively, a random library of peptides can be screened for the ability to bind the M6P/IGF-II receptor either via a yeast two hybrid assay, or via a phage display type assay.

The GILT tag can be fused to the N-terminus or C-terminus of the GAA polypeptide. The GILT tag can be fused directly to the GAA polypeptide or can be separated from the GAA polypeptide by a linker or a spacer. An amino acid linker incorporates an amino acid sequence other than that appearing at that position in the natural protein and is generally designed to be flexible or to interpose a structure, such as an alpha-helix, between the two protein moieties. A linker can be relatively short, such as the sequence Gly-Ala-Pro or Gly-Gly-Gly-Gly-Gly-Pro, or can be longer, such as, for example, 10-25 amino acids in length. The site of a fusion junction should be selected with care to promote proper folding and activity of both fusion partners and to prevent premature separation of a peptide tag from a GAA polypeptide. In a preferred embodiment, the linker sequence is Gly-Ala-Pro.

Additional constructs of GILT-tagged GAA proteins that can be used in the methods and compositions of the present invention were described in detail in U.S. Publication No. 20050244400, the entire disclosure of which is incorporated herein by reference.

GILT-tagged GAA can be expressed in a variety of mammalian cell lines including, but not limited to, human embryonic kidney (HEK) 293, Chinese hamster ovary (CHO), monkey kidney (COS), HT1080, C10, HeLa, baby hamster kidney (BHK), 3T3, C127, CV-1, HaK, NS/O, and L-929 cells. GILT-tagged GAA can also be expressed in a variety of non-mammalian host cells such as, for example, insect (e.g., Sf-9, Sf-21, Hi5), plant (e.g., Leguminosa, cereal, or tobacco), yeast (e.g., S. cerivisae, P. pastoris), prokaryote (e.g., E. coli, B. subtilis and other Bacillus spp., Pseudomonas spp., Streptomyces spp), or fungus.

In some embodiments, GILT-tagged GAA can be produced using a secretory signal peptide to facilitate secretion of the fusion protein. For example, GILT-tagged GAA can be produced using an IGF-II signal peptide. In general, the GILT-tagged GAA produced using an IGF-II signal peptide has reduced mannose-6-phophate (M6P) level on the surface of the protein compared to wild-type human GAA. As shown in the Example section, it has been confirmed by both N-linked oligosaccharide analysis and functional uptake assay that there is no detectable M6P present on an exemplary therapeutic fusion protein of the present invention.

The GILT-GAA of the present invention typically has a specific enzyme activity in the range of about 150,000-600,000 nmol/hour/mg protein, preferably in the range of about 250,000-500,000 nmol/hour/mg protein. In one embodiment, the GAA has a specific enzyme activity of at least about 150,000 nmol/hour/mg protein; preferably, a specific enzyme activity of at least about 300,000 nmol/hour/mg protein; more preferably, a specific enzyme activity of at least about 400,000 nmol/hour/mg; and even more preferably, a specific enzyme activity of at least about 600,000 nmol/hour/mg protein. GAA activity is defined by GAA 4 MU units.

### Treatment of Pompe Disease

The methods of the present invention are equally effective in treating individuals affected by infantile-, juvenile- or adult-onset Pompe disease. Typically, the therapeutic methods and compositions described herein may be more effective in treating individuals with juvenile- or adult-onset Pompe disease because these individuals have higher levels of residual GAA activity (1-10% or 10-40%, respectively), and therefore are likely to be more immunologically tolerant of the administered GILT-tagged GAA. Without wishing to be bound by theory, these patients are generally Cross-Reactive Immunologic Material (CRIM)-positive for endogenous GAA. Therefore, their immune systems likely do not perceive the GAA portion of the GILT-tagged GAA as a “foreign” protein, and are not likely to develop antibodies against the GAA portion of the GILT-tagged GAA.

The terms, “treat” or “treatment,” as used herein, refers to amelioration of one or more symptoms associated with the disease, prevention or delay of the onset of one or more symptoms of the disease, and/or lessening of the severity or frequency of one or more symptoms of the disease. For example, treatment can refer to improvement of cardiac status (e.g., increase of end-diastolic and/or end-systolic volumes, or reduction, amelioration or prevention of the progressive cardiomyopathy that is typically found in Pompe disease) or of pulmonary function (e.g., increase in crying vital capacity over baseline capacity, and/or normalization of oxygen desaturation during crying); improvement in neurodevelopment and/or motor skills (e.g., increase in AIMS score); reduction of glycogen levels in tissue of the individual affected by the disease; or any combination of these effects. In one preferred embodiment, treatment includes improvement of glycogen clearance, particularly in reduction or prevention of Pompe disease-associated cardiomyopathy. The terms, “improve,” “increase” or “reduce,” as used herein, indicate values that are relative to a baseline measurement, such as a measurement in the same individual prior to initiation of the treatment described herein, or a measurement in a control individual (or multiple control individuals) in the absence of the treatment described herein. A “control individual” is an individual afflicted with the same form of Pompe disease (either infantile, juvenile or adult-onset) as the individual being treated, who is about the same age as the individual being treated (to ensure that the stages of the disease in the treated individual and the control individual(s) are comparable).

The individual (also referred to as “patient” or “subject”) being treated is an individual (fetus, infant, child, adolescent, or adult human) having Pompe disease (i.e., either infantile-, juvenile-, or adult-onset Pompe disease) or having the potential to develop Pompe disease. The individual can have residual endogenous GAA activity, or no measurable activity. For example, the individual having Pompe disease can have GAA activity that is less than about 1% of normal GAA activity (i.e., GAA activity that is usually associated with infantile-onset Pompe disease), GAA activity that is about 1-10% of normal GAA activity (i. e., GAA activity that is usually associated with juvenile-onset Pompe disease), or GAA activity that is about 10-40% of normal GAA activity (i.e., GAA activity that is usually associated with adult-onset Pompe disease). The individual can be CRIM-positive or CRIM-negative for endogenous GAA. In one embodiment, the individual is CRIM-positive for endogenous GAA. In another embodiment, the individual is an individual who has been recently diagnosed with the disease. Early treatment (treatment commencing as soon as possible after diagnosis) is important to minimize the effects of the disease and to maximize the benefits of treatment.

### Administration of GILT-Tagged GAA

In the methods of the invention, the GILT-tagged GAA is typically administered to the individual alone, or in compositions or medicaments comprising the GILT-tagged GAA (e.g., in the manufacture of a medicament for the treatment of the disease), as described herein. The compositions can be formulated with a physiologically acceptable carrier or excipient to prepare a pharmaceutical composition. The carrier and composition can be sterile. The formulation should suit the mode of administration.

Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, sugars such as mannitol, sucrose, or others, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents (e.g., lubricants, preservatives, stabilizers, wetting agents, emulsifiers, salts for influencing osmotic pressure, buffers, coloring, flavoring and/or aromatic substances and the like) which do not deleteriously react with the active compounds or interference with their activity. In a preferred embodiment, a water-soluble carrier suitable for intravenous administration is used.

The composition or medicament, if desired, can also contain minor amounts of wetting or emulsifying agents, or pH buffering agents. The composition can be a liquid solution, suspension, emulsion, tablet, pill, capsule, sustained release formulation, or powder. The composition can also be formulated as a suppository, with traditional binders and carriers such as triglycerides. Oral formulation can include standard carriers such as pharmaceutical grades of mannitol, lactose, starch, magnesium stearate, polyvinyl pyrollidone, sodium saccharine, cellulose, magnesium carbonate, etc.

The composition or medicament can be formulated in accordance with the routine procedures as a pharmaceutical composition adapted for administration to human beings. For example, in a preferred embodiment, a composition for intravenous administration typically is a solution in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration.

The GILT-tagged GAA can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

GILT-tagged GAA (or a composition or medicament containing GILT-tagged GAA) is administered by any appropriate route. In a preferred embodiment, GILT-tagged GAA is administered intravenously. In other embodiments, GILT-tagged GAA is administered by direct administration to a target tissue, such as heart or muscle (e.g., intramuscular), or nervous system (e.g., direct injection into the brain; intraventricularly; intrathecally). Alternatively, GILT-tagged GAA (or a composition or medicament containing GILT-tagged GAA) can be administered parenterally, transdermally, or transmucosally (e.g., orally or nasally). More than one route can be used concurrently, if desired.

GILT-tagged GAA (or composition or medicament containing GILT-tagged GAA) can be administered alone, or in conjunction with other agents, such as antihistamines (e.g., diphenhydramine) or immunosuppressants or other immunotherapeutic agents which counteract anti-GILT-tagged GAA antibodies. The term, “in conjunction with,” indicates that the agent is administered prior to, at about the same time as, or following the GILT-tagged GAA (or composition containing GILT-tagged GAA). For example, the agent can be mixed into a composition containing GILT-tagged GAA, and thereby administered contemporaneously with the GILT-tagged GAA; alternatively, the agent can be administered contemporaneously, without mixing (e.g., by “piggybacking” delivery of the agent on the intravenous line by which the GILT-tagged GAA is also administered, or vice versa). In another example, the agent can be administered separately (e.g., not admixed), but within a short time frame (e.g., within 24 hours) of administration of the GILT-tagged GAA. In one preferred embodiment, if the individual is CRIM-negative for endogenous GAA, GILT-tagged GAA (or composition containing GILT-tagged GAA) is administered in conjunction with an immunosuppressive or immunotherapeutic regimen designed to reduce amounts of, or prevent production of, anti-GILT-tagged GAA antibodies. For example, a protocol similar to those used in hemophilia patients (Nilsson et al. (1988) N. Engl. J. Med., 318:947-50) can be used to reduce anti-GILT-tagged GAA antibodies. Such a regimen can also be used in individuals who are CRIM-positive for endogenous GAA but who have, or are at risk of having, anti-GILT-tagged GAA antibodies. In a particularly preferred embodiment, the immunosuppressive or immunotherapeutic regimen is begun prior to the first administration of GILT-tagged GAA, in order to minimize the possibility of production of anti-GILT-tagged GAA antibodies.

GILT-tagged GAA (or composition or medicament containing GILT-tagged GAA) is administered in a therapeutically effective amount (i.e., a dosage amount that, when administered at regular intervals, is sufficient to treat the disease, such as by ameliorating symptoms associated with the disease, preventing or delaying the onset of the disease, and/or also lessening the severity or frequency of symptoms of the disease, as described above). The dose which will be therapeutically effective for the treatment of the disease will depend on the nature and extent of the disease's effects, and can be determined by standard clinical techniques. In addition, in vitro or in vivo assays may optionally be employed to help identify optimal dosage ranges, such as those exemplified below. The precise dose to be employed will also depend on the route of administration, and the seriousness of the disease, and should be decided according to the judgment of a practitioner and each patient's circumstances. Effective doses may be extrapolated from dose-response curves derived from in vitro or animal model test systems. The therapeutically effective dosage amount can be, for example, about 0.1-1 mg/kg, about 1-5 mg/kg, about 5-20 mg/kg, about 20-50 mg/kg, or 20-100 mg/kg. The effective dose for a particular individual can be varied (e.g., increased or decreased) over time, depending on the needs of the individual. For example, in times of physical illness or stress, or if anti-GILT-tagged GAA antibodies become present or increase, or if disease symptoms worsen, the dosage amount can be increased.

The therapeutically effective amount of GILT-tagged GAA (or composition or medicament containing GILT-tagged GAA) is administered at regular intervals, depending on the nature and extent of the disease's effects, and on an ongoing basis. Administration at an “interval,” as used herein, indicates that the therapeutically effective amount is administered periodically (as distinguished from a one-time dose). The interval can be determined by standard clinical techniques. In preferred embodiments, GILT-tagged GAA is administered bimonthly, monthly, twice monthly, triweekly, biweekly, weekly, twice weekly, thrice weekly, or daily. The administration interval for a single individual need not be a fixed interval, but can be varied over time, depending on the needs of the individual. For example, in times of physical illness or stress, if anti-GILT-tagged GAA antibodies become present or increase, or if disease symptoms worsen, the interval between doses can be decreased.

As used herein, the term “bimonthly” means administration once per two months (i.e., once every two months); the term “monthly” means administration once per month; the term “triweekly” means administration once per three weeks (i.e., once every three weeks); the term “biweekly” means administration once per two weeks (i.e., once every two weeks); the term “weekly” means administration once per week; and the term “daily” means administration once per day.

The invention additionally pertains to a pharmaceutical composition comprising human GILT-tagged GAA, as described herein, in a container (e.g., a vial, bottle, bag for intravenous administration, syringe, etc.) with a label containing instructions for administration of the composition for treatment of Pompe disease, such as by the methods described herein.

The invention will be further and more specifically described by the following examples. Examples, however, are included for illustration purposes, not for limitation.

### EXAMPLES

### Example 1

### Production of Recombinant Wild-Type GAA and GILT-Tagged GAA

### Plasmids

DNA encoding full-length, wild-type human GAA was isolated and inserted into an expression vector for production of recombinant human GAA. A DNA cassette encoding complete human GAA amino acids 1-952 (hereinafter “cassette 635”) was derived from IMAGE clone 4374238 (Open Biosystems) using the following PCR primers:

(SEQ ID NO:1)

GAA13: 5′-GGAATTCCAACCATGGGAGTGAGGCACCCGCCC
and

(SEQ ID NO:2)

GAA27: 5′-GCTCTAGACTAACACCAGCTGACGAGAAACTGC.

Cassette 635 was digested with EcoRI and XbaI, blunted by treatment with Klenow DNA polymerase, then ligated into the Klenow-treated HindIII site of expression vector pCEP4 (Invitrogen) to create plasmid p635. Hereinafter, ZC-635 refers to wild-type, untagged GAA protein.

A DNA cassette for the production of recombinant GILT-tagged GAA ZC-701 (hereinafter “cassette 701”) was prepared similarly to cassette 635, except for the following N-terminal sequence that was joined upstream of GAA sequence corresponding to amino acid A70:

(SEQ ID NO:3)

GAATTCACACCAATGGGAATCCCAATGGGGAAGTCGATGCTGGTGCTTCT
CACCTTCTTGGCCTTCGCCTCGTGCTGCATTGCTGCTCTGTGCGGCGGGG
AGCTGGTGGACACCCTCCAGTTCGTCTGTGGGGACCGCGGCTTCTACTTC
AGCAGGCCCGCAAGCCGTGTGAGCCGTCGCAGCCGTGGCATCGTTGAGGA
GTGCTGTTTCCGCAGCTGTGACCTGGCCCTCCTGGAGACGTACTGTGCTA
CCCCCGCCAAGTCCGAGGGCGCGCCG.

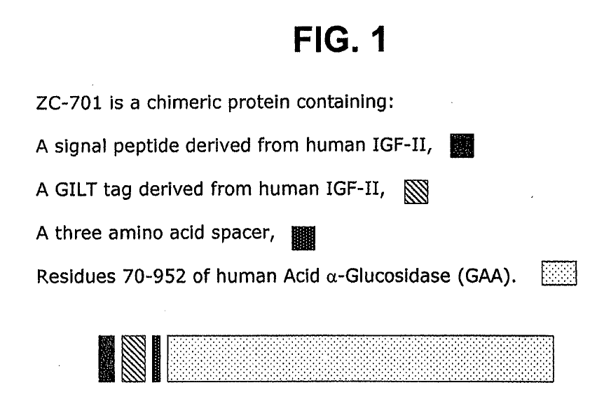

Cassette 701 was digested with EcoRI and XbaI, blunted by treatment with Klenow DNA polymerase, then ligated into the Klenow-treated HindIII site of expression vector pCEP4 to create plasmid p701. Hereinafter, ZC-701 refers to GILT-tagged GAA protein encoded by the p701 plasmid. FIG. 1 shows a diagram of GILT-tagged GAA ZC-701, including the IGF-II signal peptide which would be lost upon secretion. Thus, in secreted form (i.e., as it would be administered to a subject), ZC-701 includes amino acids 1 and 8-67 of human IGF-II (i.e., Δ2-7 of mature human IGF-II), the spacer sequence Gly-Ala-Pro, and amino acids 70-952 of human GAA. The full length amino acid sequence is shown below. The spacer sequence is underlined. The sequence N-terminal to the spacer sequence reflects amino acids 1 and 8-67 of human IGF-II (arrow points to amino acid 1) and the sequence C-terminal to the spacer sequence reflects amino acids 70-952 of human GAA.

(SEQ ID NO:4)

↓
MGIPMGKSMLVLLTFLAFASCCIAALCGGELVDTLQFVCGDRGFYFSRPA
SRVSRRSRGIVEECCFRSCDLALLETYCATPAKSEGAPAHPGRPRAVPTQ
CDVPPNSRFDCAPDKAITQEQCEARGCCYIPAKQGLQGAQMGQPWCFFPP
SYPSYKLENLSSSEMGYTATLTRTTPTFFPKDILTLRLDVMMETENRLHF
TIKDPANRRYEVPLETPRVHSRAPSPLYSVEFSEEPFGVIVHRQLDGRVL
LNTTVAPLFFADQFLQLSTSLPSQYITGLAEHLSPLMLSTSWTRITLWNR
DLAPTPGANLYGSHPFYLALEDGGSAHGVFLLNSNAMDVVLQPSPALSWR
STGGILDVYIFLGPEPKSVVQQYLDVVGYPFMPPYWGLGFHLCRWGYSST
AITRQVVENMTRAHFPLDVQWNDLDYMDSRRDFTFNKDGFRDFPAMVQEL
HQGGRRYMMIVDPAISSSGPAGSYRPYDEGLRRGVFITNETGQPLIGKVW
PGSTAFPDFTNPTALAWWEDMVAEFHDQVPFDGMWIDMNEPSNFIRGSED
GCPNNELENPPYVPGVVGGTLQAATICASSHQFLSTHYNLHNLYGLTEAI
ASHRALVKARGTRPFVISRSTFAGHGRYAGHWTGDVWSSWEQLASSVPEI
LQFNLLGVPLVGADVCGFLGNTSEELCVRWTQLGAFYPFMRNHNSLLSLP
QEPYSFSEPAQQAMRKALTLRYALLPHLYTLFHQAHVAGETVARPLFLEF
PKDSSTWTVDHQLLWGEALLITPVLQAGKAEVTGYFPLGTWYDLQTVPIE
ALGSLPPPPAAPREPAIHSEGQWVTLPAPLDTINVHLRAGYIIPLQGPGL
TTTESRQQPMALAVALTKGGEARGELFWDDGESLEVLERGAYTQVIFLAR
NNTIVNELVRVTSEGAGLQLQKVTVLGVATAPQQVLSNGVPVSNFTYSPD
TKVLDICVSLLMGEQFLVSWC

A second GILT-tagged GAA cassette, ZC-1026, was constructed similarly. ZC-1026 includes amino acids 1 and 8-67 of human IGF-II, the spacer sequence Thr-Gly, and amino acids 70-952 of human GAA.

These plasmids were used to transiently transfect suspension HEK293 cells for production of recombinant proteins. Plasmids were transfected into suspension FreeStyle™ 293-F cells as described by the manufacturer (Invitrogen). Briefly, cells were grown in Opti-MEM® I media (Invitrogen) in polycarbonate shaker flasks on an orbital shaker at 37° C. and 8% CO₂. Cells were adjusted to a concentration of 1×10⁶ cells/ml, then transfected with a 1:1:1 ratio of ml cells:μg DNA:μl 293fectin™ as described by the manufacturer (Invitrogen). Cultures were harvested 5-10 days post transfection and cells were removed by centrifugation and filtration through 0.2 μm bottle-top filters. Supernatants were stored at −80° C.

Alternatively, cassette 701 was incorporated into the GPEx® retrovector expression system (Cardinal Health). The process was described in U.S. Pat. No. 6,852,510, the disclosure of which is hereby incorporated by reference. The GPEx® retrovector expression system containing cassette 701 was used to create a stable CHO cell line for production of recombinant GILT-tagged GAA. Cassette 701 can also be incorporated into the GPEX® retrovector expression system and used to create a stable HEK293 cell line for the production of recombinant GILT-tagged GAA.

### Purification of GILT-Tagged GAA

Starting material was mammalian cell culture supernatant, as described above, thawed from storage at −80 C. Sodium acetate (pH 4.6) was added to reach the final concentration of 100 mM and ammonium sulfate was added to reach the final concentration of 0.75M. The material was centrifuged to remove precipitation and filtered with a 0.8/0.2 μm AcroPak™ 500 capsule (Pall, catalog #12991).

The filtered material was loaded onto a Phenyl-Sepharose™ Low-Sub Fast-Flow (GE Healthcare) column prepared with HIC Load Buffer (50 mM NaAc pH 4.6, 0.75M AmSO₄). The column was washed with 10 column volumes of HIC Wash Buffer (50 mM NaAc pH 5.3, 0.75M AmSO₄) and eluted with 5 column volumes of HIC Elution Buffer (50 mM NaAc pH 5.3, 20 mM AmSO₄).

Pooled fractions were extensively dialyzed into QXL load buffer (20 mM Histidine pH 6.5, 50 mM NaCl) then loaded onto a Q Sepharose™ XL column (GE Healthcare). The column was washed with 10 column volumes of QXL Equilibration Buffer, and eluted with 10 column volumes of QXL Elution Buffer (20 mM Histidine pH 6.5, 150 mM NaCl). In some cases, pooled fractions from the QXL column were concentrated to a protein concentration between 30 and 40 mg/ml then loaded onto a 2.6×90 cm Ultrogel® AcA 44 column equilibrated in PBS pH 6.2. Loading volumes were between 5 and 7.5 ml (1-1.5% of the column volume). The column was run in PBS pH 6.2 at 0.4 ml/min and 4 ml fractions were collected.

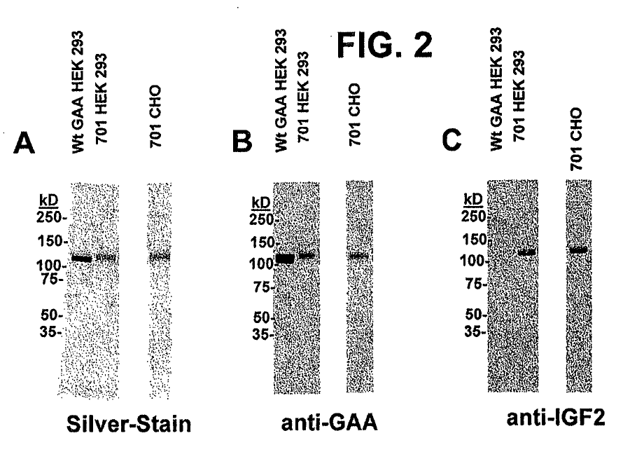

The purified untagged GAA and GILT-tagged GAA are shown in FIGS. 2A-C. FIG. 2A shows SDS-PAGE followed by silver staining. FIG. 2B shows a Western blot using anti-GAA antibody. FIG. 2C shows a Western blot using anti-IGF-II antibody.

### Example 2

### Affinity of GILT-Tagged GAA for the CI-MPR

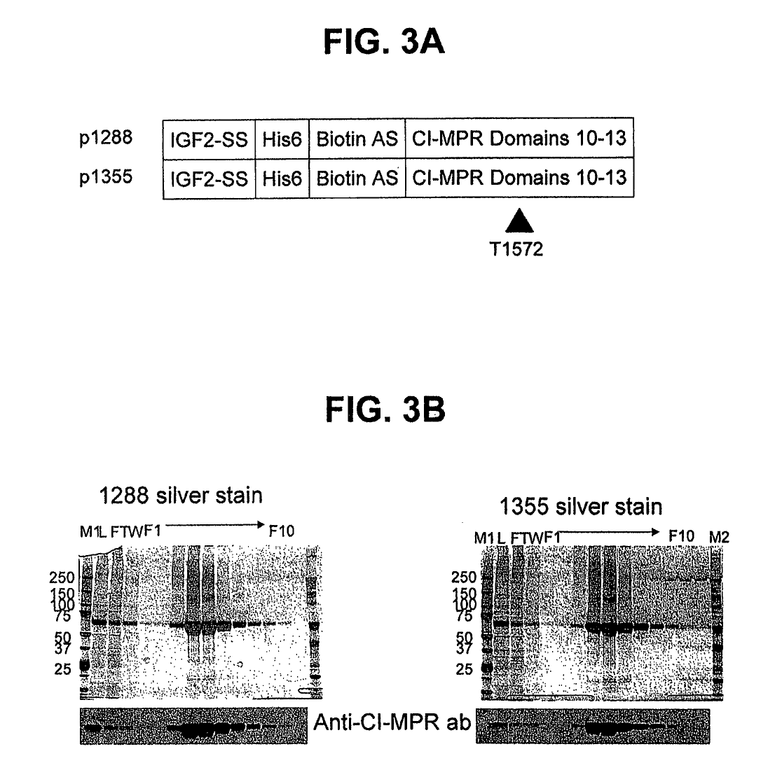

The binding affinity of GILT-tagged GAA ZC-701 for the CI-MPR was determined using a Biacore® surface plasmon resonance assay. Two biotinylated and His-tagged recombinant proteins containing wild-type CI-MPR domains 10-13 and a point mutant variant, respectively, were made according to standard molecular techniques. Schematic representations of the two recombinant proteins are shown in FIG. 3A. Plasmid p1288 contains an IGF-II signal peptide followed by: a poly-His tag; a Biotin AS domain; and a sequence encoding wild-type CI-MPR domains 10-13. Plasmid p1355 contains an IGF-II signal peptide followed by: a poly-His tag; a Biotin AS domain; and a sequence encoding CI-MPR domains 10-13 with a point mutation 11572T that effectively decreases the affinity of the receptor for IGF-II. Specific DNA and amino acid sequences relating to the two recombinant proteins are shown below.

(SEQ ID NO:5)

HIS-BIOTIN-CI-MPR DOMAINS 10-13
ATGGGAATCCCAATGGGGAAGTCCATGCTGGTGCTTCTCACCTTCTTGGC
CTTCGCCTCGTGCTGCATTGCTGCTGGCGCGCCGACCGGTCACCATCACC
ATCACCACGCGCCGGGCCTGAACGACATCTTCGAGGCCCAGAAGATCGAG
TGGCACGAACCTTTCGATCTGACTGAATGTTCATTCAAAGATGGGGCTGG
CAACTCCTTCGACCTCTCGTCCCTGTCAAGGTACAGTGACAACTGGGAAG
CCATCACTGGGACGGGGGACCCGGAGCACTACCTCATCAATGTCTGCAAG
TCTCTGGCCCCGCAGGCTGGCACTGAGCCGTGCCCTCCAGAAGCAGCCGC
GTGTCTGCTGGGTGGCTCCAAGCCCGTGAACCTCGGCAGGGTAAGGGACG
GACCTCAGTGGAGAGATGGCATAATTGTCCTGAAATACGTTGATGGCGAC
TTATGTCCAGATGGGATTCGGAAAAAGTCAACCACCATCCGATTCACCTG
CAGCGAGAGCCAAGTGAACTCCAGGCCCATGTTCATCAGCGCCGTGGAGG
ACTGTGAGTACACCTTTCCCTGGCCCACAGCCACAGCCTGTCCCATGAAG
AGCAACGAGCATGATGACTGCCAGGTCACCAACCCAAGCACAGGACACCT
GTTTGATCTGAGCTCCTTAAGTGGCAGGGCGGGATTCACAGCTGCTTACA
GCGAGAAGGGGTTGGTTTACATGAGCATCTGTGGGGAGAATGAAAACTGC
CCTCCTGGCGTGGGGGCCTGCTTTGGACAGACCAGGATTAGCGTGGGCAA
GGCCAACAAGAGGCTGAGATACGTGGACCAGGTCCTGCAGCTGGTGTACA
AGGATGGGTCCCCTTGTCCCTCCAAATCCGGCCTGAGCTATAAGAGTGTG
ATCAGTTTCGTGTGCAGGCCTGAGCCCGGGCCAACCAATAGGCCCATGCT
CATCTCCCTGGACAAGCAGACATGCACTCTCTTCTTCTCCTGGCACACGC
CGCTGGCCTGCGAGCAAGCGACCGAATGTTCCGTGAGGAATGGAAGCTCT
ATTGTTGACTTGTCTCCCCTTATTCATCGCACTGGTGGTTATGAGGCTTA
TGATGAGAGTGAGGATGATGCCTCCGATACCAACCCTGATTTCTACATCA
ATATTTGTCAGCCACTAAATCCCATGCACGGAGTGCCCTGTCCTGCCGGA
GCCGCTGTGTGCAAAGTTCCTATTGATGGTCCCCCCATAGATATCGGCCG
GGTAGCAGGACCACCAATACTCAATCCAATAGCAAATGAGATTTACTTGA
ATTTTGAAAGCAGTACTCCTTGCTTAGCGGACAAGCATTTCAACTACACC
TCGCTCATCGCGTTTCACTGTAAGAGAGGTGTGAGCATGGGAACGCCTAA
GCTGTTAAGGACCAGCGAGTGCGACTTTGTGTTCGAATGGGAGACTCCTG
TCGTCTGTCCTGATGAAGTGAGGATGGATGGCTGTACCCTGACAGATGAG
CAGCTCCTCTACAGCTTCAACTTGTCCAGCCTTTCCACGAGCACCTTTAA
GGTGACTCGCGACTCGCGCACCTACAGCGTTGGGGTGTGCACCTTTGCAG
TCGGGCCAGAACAAGGAGGCTGTAAGGACGGAGGAGTCTGTCTGCTCTCA
GGCACCAAGGGGGCATCCTTTGGACGGCTGCAATCAATGAAACTGGATTA
CAGGCACCAGGATGAAGCGGTCGTTTTAAGTTACGTGAATGGTGATCGTT
GCCCTCCAGAAACCGATGACGGCGTCCCCTGTGTCTTCCCCTTCATATTC
AATGGGAAGAGCTACGAGGAGTGCATCATAGAGAGCAGGGCGAAGCTGTG
GTGTAGCACAACTGCGGACTACGACAGAGACCACGAGTGGGGCTTCTGCA
GACACTCAAACAGCTACCGGACATCCAGCATCATATTTAAGTGTGATGAA
GATGAGGACATTGGGAGGCCACAAGTCTTCAGTGAAGTGCGTGGGTGTGA
TGTGACATTTGAGTGGAAAACAAAAGTTGTCTGCCCTTGA
HIS-BIOTIN-CI-MPR DOMAINS 10-13 PROTEIN SEQUENCE

(SEQ ID NO:6)

MGIPMGKSMLVLLTFLAFASCCIAAGAPTGHHHHHHAPGLNDIFEAQKIE
WHEPFDLTECSFKDGAGNSFDLSSLSRYSDNWEAITGTGDPEHYLINVCK
SLAPQAGTEPCPPEAAACLLGGSKPVNLGRVRDGPQWRDGIIVLKYVDGD
LCPDGIRKKSTTIRFTCSESQVNSRPMFISAVEDCEYTFAWPTATACPMK
SNEHDDCQVTNPSTGHLFDLSSLSGRAGFTAAYSEKGLVYMSICGENENC
PPGVGACFGQTRISVGKANKRLRYVDQVLQLVYKDGSPCPSKSGLSYKSV
ISFVCRPEAGPTNRPMLISLDKQTCTLFTSWHTPLACEQATECSVRNGSS
IVDLSPLIHRTGGYEAYDESEDDASDTNPDFYINICQPLNPMHGVPCPAG
AAVCKVPIDGPPIDIGRVAGPPILNPIANEIYLNFESSTPCLADKHFNYT
SLIAFHCKRGVSMGTPKLLRTSECDFVFEWETPVVCPDEVRMDGCTLTDE
QLLYSFNLSSLSTSTFKVTRDSRTYSVGVCTFAVGPEQGGCKDGGVCLLS
GTKGASFGRLQSMKLDYRHQDEAVVLSYVNGDRCPPETDDGVPCVEPFIF
NGKSYEECIIESRAKLWCSTTADYDRDHEWGFCRHSNSYRTSSIIFKCDE
DEDIGRPQVFSEVRGCDVTFEWKTKVVCP.
HIS-BIOTIN-CI-MPR DOMAINS 10-13 11572T
(the underlined sequence change results in the
point mutation I1572T and a silent mutation S1573
that creates a diagnostic SpeI site)

(SEQ ID NO:7)

ATGGGAATCCCAATGGGGAAGTCGATGCTGGTGCTTCTCACCTTCTTGGC
CTTCGCCTCGTGCTGCATTGCTGCTGGCGCGCCGACCGGTCACCATCACC
ATCACCACGCGCCGGGCCTGAACGACATCTTCGAGGCCCAGAAGATCGAG
TGGCACGAACCTTTCGATCTGACTGAATGTTCATTCAAAGATGGGGCTGG
CAACTCCTTCGACCTCTCGTCCCTGTCAAGGTACAGTGACAACTGGGAAG
CCATCACTGGGACGGGGGACCCGGAGCACTACCTCATCAATGTCTGCAAG
TCTCTGGCCCCGCAGGCTGGCACTGAGCCGTGCCCTCCAGAAGCAGCCGC
GTGTCTGCTGGGTGGCTCCAAGCCCGTGAACCTCGGCAGGGTAAGGGACG
GACCTCAGTGGAGAGATGGCATAATTGTCCTGAAATACGTTGATGGCGAC
TTATGTCCAGATGGGATTCGGAAAAAGTCAACCACCATCCGATTCACCTG
CAGCGAGAGCCAAGTGAACTCCAGGCCCATGTTCATCAGCGCCGTGGAGG
ACTGTGAGTACACCTTTGCCTGGCCCACAGCCACACCCTGTCCCATGAAG
AGCAACGAGCATGATGACTGCCAGGTCACCAACCCAAGCACAGGACACCT
GTTTGATCTGAGCTCCTTAAGTCGCAGGGCGGGATTCACAGCTGCTTACA
GCGAGAAGGGGTTGGTTTACATGAGCATCTGTGGGGAGAATGAAAACTGC
CCTCCTGGCGTGGGGGCCTGCTTTGGACAGACCAGGACTAGTGTGGGCAA
GGCCAACAAGAGGCTGAGATACGTGGACCAGGTCCTGCAGCTGGTGTACA
AGGATGGGTCCCCTTGTCCCTCCAAATCCGGCCTGAGCTATAAGAGTGTG
ATCAGTTTCGTGTGCAGGCCTGAGGCCGGGCCAACCAATAGGCCCATGCT
CATCTCCCTGGACAAGCAGACATGCACTCTCTTCTTCTCCTGGCACACGC
CGCTGGCCTGCGAGCAAGCGACCGAATGTTCCGTGAGGAATGGAAGCTCT
ATTGTTGACTTGTCTCCCCTTATTCATCGCACTGGTGGTTATGAGGCTTA
TGATGAGAGTGAGGATGATGCCTCCGATACCAACCCTGATTTCTACATCA
ATATTTGTCAGCCACTAAATCCCATGCACGGAGTGCCCTGTCCTGCCGGA
GCCGCTGTGTGCAAAGTTCCTATTGATGGTCCCCCCATAGATATCGGCCG
GGTAGCAGGACCACCAATACTCAATCCAATAGCAAATGAGATTTACTTGA
ATTTTGAAAGCAGTACTCCTTGCTTAGCGGACAAGCATTTCAACTACACC
TCGCTCATCGCGTTTCACTGTAAGAGAGGTGTGAGCATGGGAACGCCTAA
GCTGTTAAGGACCAGCGAGTGCGACTTTCTGTTCGAATGGGAGACTCCTG
TCGTCTGTCCTGATGAAGTGAGGATGGATGGCTGTACCCTGACAGATGAG
CAGCTCCTCTACAGCTTCAACTTGTCCAGCCTTTCCACGAGCACCTTTAA
GGTGACTCGCCACTCGCGCACCTACAGCGTTGGGGTGTGCACCTTTGCAG
TCGGGCCAGAACAAGGAGGCTGTAAGGACGGAGGAGTCTGTCTGCTCTCA
GGCACCAAGGGGGCATCCTTTGGACGGCTGCAATCAATGAAACTGGATTA
CAGGCACCAGGATGAAGCGGTCGTTTTAAGTTACGTGAATGGTGATCGTT
GCCCTCCAGAAACCGATGACGGCGTCCCCTGTGTCTTCCCCTTCATATTC
AATGGGAAGAGCTACGAGGAGTGCATCATAGAGAGCAGGGCGAAGCTGTG
GTGTAGCACAACTGCGGACTACGACAGAGACCACGAGTGGGGCTTCTGCA
GACACTCAAACAGCTACCGGACATCCAGCATCATATTTAAGTGTGATGAA
GATGAGGACATTGGGAGGCCACAAGTCTTCAGTGAAGTGCGTGGGTGTGA
TGTGACATTTGAGTGGAAAACAAAAGTTGTCTGCCCTTGA
HIS-BIOT1N-CI-MPR DOMAINS 10-13 I1572T PROTEIN
SEQUENCE (the I1572T mutation is underlined)

(SEQ ID NO:8)

MGIPMGKSMLVLLTFLAFASCCIAAGAPTGHHHHHHAPGLNDIFEAQKIE
WHEPFDLTECSFKDGAGNSFDLSSLSRYSDNWEAITGTGDPEHYLINVCK
SLAPQAGTEPCPPEAAACLLGGSKPVNLGRVRDGPQWRDGIIVLKYVDGD
LCPDGIRKKSTTIRFTCSESQVNSRPMFISAVEDCEYTFAWPTATACPMK
SNEHDDCQVTNPSTGHLFDLSSLSGRAGFTAAYSEKGLVYMSICGENENC
PPGVGACFGQTRTSVGKANKRLRYVDQVLQLVYKDGSPCPSKSGLSYKSV
ISFVCRPEAGPTNRPMLISLDKQTCTLFFSWHTPLACEQATECSVRNGSS
IVDLSPLIHRTGGYEAYDESEDDASDTNPDFYINICQPLNPMHGVPCPAG
AAVCKVPIDGPPIDIGRVAGPPILNPIANEIYLNFESSTPCLADKHFNYT
SLIAFHCKRGVSMGTPKLLRTSECDFVFEWETPVVCPDEVRMDGCTLTDE
QLLYSFNLSSLSTSTFKVTRDSRTYSVGVCTFAVGPEQGGCKDGGVCLLS
GTKGASFGRLQSMKLDYRHQDEAVVLSYVNGDRCPPETDDGVPCVFPFIF
NGKSYEECIIESRAKLWCSTTADYDRDHEWGFCRHSNSYRTSSIIFKCDE
DEDIGRPQVFSEVRGCDVTFEWKTKVVCP

Recombinant proteins, expressed transiently in suspension HEK293 cells (see FIG. 3B). The proteins were collected from the culture supernatant and purified by nickel agarose, and then biotinylated. Specifically, supernatant from cells transfected with plasmids p1288 and p1355 were applied to a 1 ml His Gravitrap™ column (GE Healthcare) as directed by the manufacturer for purification of the His₆-tagged receptor domain proteins. Elutions were concentrated and exchanged into 10 mM Tris pH8 and 25 mM NaCl buffer, then the proteins were biotinylated with BirA enzyme as described by the manufacturer (Avidity) in reactions that contained 70 μg receptor in 205 μl total volume with 25 μl BiomixA, 25 μl BiomixB, and 4 μl BirA enzyme. BirA enzyme treatment was performed at 30° C. for 1.5 hours. The reactions were then diluted 20 fold into His Gravitrap™ binding buffer (GE Healthcare) and re-applied to the His Gravitrap™ column for removal of BirA enzyme and free biotin. Elutions were stored at 4° C.

### Surface Plasmon Resonance Analysis

All surface plasmon resonance measurements were performed at 25° C. using a Biacore® 3000 instrument. SA sensor chips and surfactant P20 were obtained from Biacore (Piscataway, N.J.). All buffers were filtered using Nalgene® filtration units (0.2 μm), equilibrated to room temperature, and degassed immediately prior to use. Protein samples were centrifuged at 13,000×g for 15 minutes to remove any particulates that may be present in the sample.

Purified, biotinylated wild-type (1288FS) or mutant (1355FS) recombinant CI-MPR domains 10-13 (Dom10-13) proteins were immobilized on a Biacore® streptavidin (SA) chip which contains a dextran matrix to which streptavidin has been covalently attached. Following docking of the SA sensor chip, the SA chip was washed two times with deionized water. The flow cells to be coupled were conditioned by injecting 60 ml of a buffer containing 50 mM NaOH/1 M NaCl at a flow rate of 20 ml/min. The chip was washed with H2O as described above. The chip was then washed with coupling buffer (10 mM HEPES pH 7.4/100 mM NaCl) as described above. The biotinylated Dom10-13 proteins 1355FS and 1288FS were diluted to 20 ng/ml and 4 ng/ml in coupling buffer. Flow cells (FC) 1 & 2 were coupled at a higher density than FC 3 & 4. FC1 and FC3 were immobilized with the mutant Dom 10-13 construct and were used as the reference surface (i.e., the response obtained from FC1 was subtracted from FC2; the response obtained from FC3 was subtracted from FC4). FC2 and FC4 were immobilized with the wild-type Dom10-13 construct. FC1 was coupled by injecting 50 ml of 1355FS (20 ng/ml) at a flow rate of 10 ml/min and FC2 was coupled by injecting 50 ml of 1288FS (20 ng/ml) at a flow rate of 10 ml/min to reach a final coupling level of approximately 5,000 resonance units (RU). FC3 was coupled by injecting 50 ml of 1355FS (4 ng/ml) at a flow rate of 10 ml/min and FC2 was coupled by injecting 50 ml of 1288FS (4 ng/ml) at a flow rate of 10 ml/min to reach a final coupling level of approximately 1,000 RU. These coupling levels give a theoretical Rmax for IGF-II of 800 RU (FC2-1) or 160 RU (FC4-3) and a theoretical Rmax for GAA-GILT of 10,000 RU (FC2-1) or 2,000 RU (FC4-3). Following the 50 ml injection of coupling buffer containing the Dom10-13 constructs, the chip was washed with coupling buffer alone (10 ml injection at a flow rate of 10 ml/min). Remaining unbound streptavidin binding sites were saturated with biotin by two sequential 10 ml injections of biotin (10 mM) at a flow rate of 10 μl/min.

After coupling, the flow cells were equilibrated in running buffer (10 mM HEPES pH 7.0, 150 mM NaCl, and 0.005% (v/v) surfactant P20). The activity of the immobilized Dom10-13 construct was determined by measuring the affinity of the receptor to IGF-II alone. IGF-II (134 mM) was first diluted to final concentrations of 1, 5, 10, 25, 50, 75, 100, 250, and 500 nM in running buffer. Each IGF-II concentration was injected onto the chip at a flow rate of 40 ml/min for 2 min (i.e., association phase) followed by a 2 min injection (flow rate=40 ml/min) of running buffer alone (i.e., dissociation phase). The surface was regenerated with a 10 ml injection of 10 mM HCl at a flow rate of 10 ml/min. After regeneration, the flow rate was increased to 40 ml/min and the chip was allowed to equilibrate for 1 min prior to beginning the next injection.

Similarly, the GILT-tagged GAA construct 701 was assayed for its binding affinity for Dom10-13 recombinant receptors. The constructs were diluted to final concentrations of 1, 5, 10, 25, 50, 75, 100, 250, and 500 nM in running buffer and injected as described above for IGF-II. An IGF-II concentration curve was run after the GILT-tagged GAA constructs to test the integrity of the immobilized Dom10-13 surfaces.

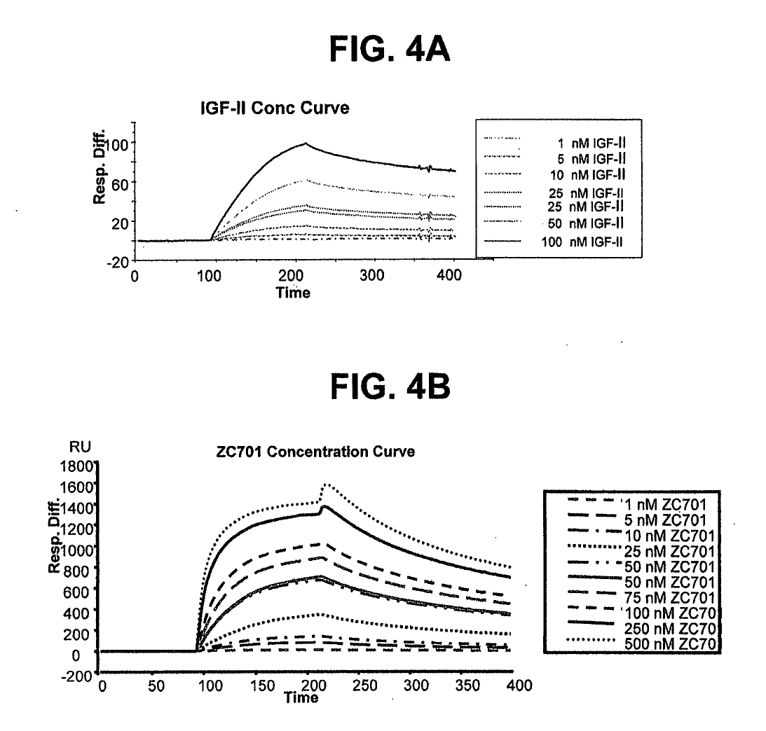

An average of the responses at equilibrium was determined for each analyte concentration, and the resulting equilibrium resonance units were plotted against concentration. Data were fit to a steady-state affinity model using BIAevaluation™ software (version 4.1). Dissociation constants were also determined using a 1:1 binding isotherm model. All response data were double-referenced as described in Myszka (2000) Methods Enzymol. 323:325-340, where controls (i.e., running buffer alone injections) for the contribution of the change in bulk refractive index were performed in parallel with flow cells immobilized with mutant Dom10-13 and subtracted from all binding sensorgrams. FIGS. 4A-B are exemplary concentration curves showing Biacore® analysis of IGF-II and GILT-tagged GAA ZC-701 binding to CI-MPR. FIG. 4A shows binding curves for IGF-II. FIG. 4B shows binding curves for GILT-tagged GAA ZC-701. Results from both flow cell pairs (i.e., FC2-1 and FC4-3) were compared (FIG. 4B).

Results of these experiments indicate that the GILT-tagged GAA ZC-701 has an affinity for the CI-MPR that is about 0.8 that of IGF-II (Table 1). These data indicate that GILT-tagged GAA has a high affinity for the CI-MPR, comparable to that of IGF-II. Although the absolute value of the measured affinity of IGF-II for the CI-MPR was 27 nM, it was previously reported in the literature that IGF-II binds to domains 10-13 of the receptor with about 10 fold lower affinity than to the native receptor. Linnell et al. (2001) J Biol Chem. June 29;276(26):23986-91. Accordingly, the binding affinity of GILT-tagged GAA for the native receptor is also expected to be 10-fold higher.

TABLE 1

| Protein | K_{d} | Relative Affinity |
|---|---|---|
| IGF-II | 27 nM | 1 |
| ZC-701 | 33 nM | 0.8 |
| ZC-1026 | 43 nM | 0.6 |

### Example 3

### N-Linked Oligosaccharide Analysis Indicates ZC-701 Lacks M6P

N-linked oligosaccharide analysis was conducted to determine oligosaccharide profiles for ZC-701, using the combination of PNGase deglycosylation followed by HPLC analysis with fluorescence detection (Blue Stream Laboratories).

Cleavage of N-linked carbohydrates from the glycoprotein samples was performed by means of N-glycanase, at a ratio of 1:100 (enzyme to substrate) using approximately 100 μg of protein for each sample. Once released, glycans were extracted using cold ethanol and brought to dryness by centrifugation. The recovered oligosaccharides were labeled with 2-aminobenzamide (2-AB) in the presence of sodium cyanoborohydride under acidic conditions. Subsequent to the derivitization step, excess dye and other reaction reagents left in the samples were removed by means of Glycoclean® S sample filtration cartridges (Prozyme).

Analysis of N-Linked oligosaccharides by HPLC-FLD using the following conditions: Mobile Phase A: 65% Acetonitrile/35% Mobile B; Mobile Phase B: 250 mM Ammonium Formate, pH 4.4; Detection: Fluorescence (Ex: 330 nm, Em: 420 nm); and HPLC Gradient.

Chromatographic peaks were integrated, and based on peak retention times, were compared. Results were reported as % area of each glycoform per total peak area. From this analysis, it was determined that the predominant oligosaccharide structures present on ZC-701 are high mannose structures and some complex antennary structures. However, no mannose-6-phosphate structure was detected.

### Example 4

### Uptake Assay Demonstrates the Functional Absence of M6P on the Surface of ZC-701

Uptake of recombinant GAA into mammalian cells is mediated by interaction with the CI-MPR, which is present on the surface of most mammalian cell types. Uptake depends upon the presence of M6P on the oligosaccharides on the protein's surface.

In contrast, ZC-701 has a high affinity for the CI-MPR due to the presence of the IGF-II derived tag at the N-terminus of the protein. In a variety of experiments, it has been shown that ZC-701 displays no appreciable M6P-dependent uptake into mammalian cells, which demonstrates the functional absence of M6P on the surface of ZC-701.

Cell-based uptake assays were performed to demonstrate the ability of GILT-tagged or untagged GAA to enter the target cell. Rat L6 myoblasts were plated at a density of 1×105 cells per well in 24-well plates 24 hours prior to uptake. At the start of the experiment, the media was removed from the cells and replaced with 0.5 ml of uptake media which contains tagged-or untagged GAA ranging in concentrations from 2-500 nM. In order to demonstrate specificity of uptake, some wells additionally contained the competitors M6P (5mM final concentration) and/or IGF-II (18 μg/ml final concentration). After 18 hours, media was aspirated off of cells, and cells were washed 4 times with PBS. Then, cells were lysed with 200 μl CelLytic MTM lysis buffer. The lysate was assayed for GAA activity as described below using the 4 MU substrate. Protein was determined using the Pierce BCATM Protein Assay Kit.

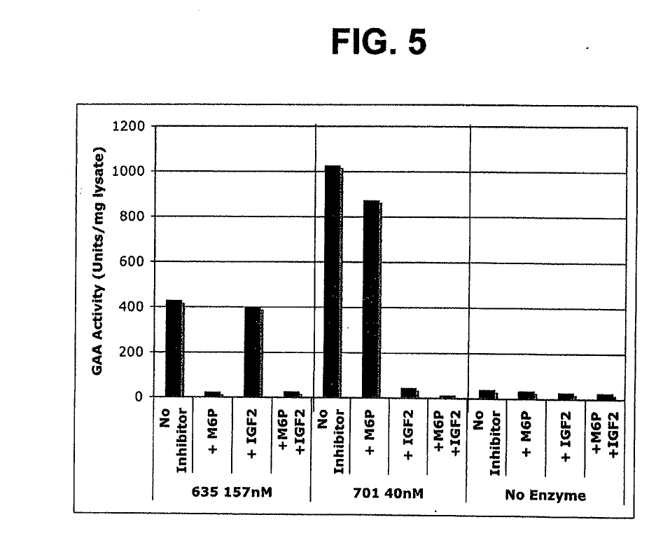

A typical uptake experiment result for ZC-701 produced in CHO cells is shown in FIG. 5. As can be seen, uptake of ZC-701 into Rat L6 myoblasts was virtually unaffected by the addition of a large molar excess of M6P, whereas uptake was completely abolished by excess IGF-II. In contrast, uptake of wtGAA (ZC-635) was completely abolished by addition of excess M6P but virtually unaffected by competition with IGF-II. The insensitivity of CHO-cell produced ZC-701 uptake into mammalian cells to inhibition by excess M6P indicates the functional absence of M6P on the surface of ZC-701 produced in CHO cells.

### Example 5

### GILT-Tagged GAA Shows More Efficient Uptake Than Untagged GAA

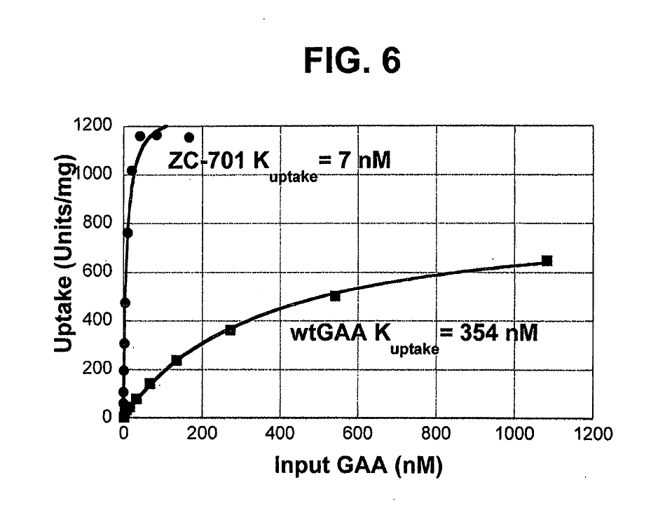

FIG. 6 shows saturation curves for uptake into L6 Myoblasts of purified GILT-tagged GAA (ZC-701) and wild-type, untagged GAA (ZC-635). In the illustrated experiment, GILT-tagged GAA has a Kuptake=7 nM whereas wt GAA has a Kuptake=354 nM. This indicates that GILT-tagged GAA shows more efficient uptake than untagged GAA because significantly lower levels of GILT-tagged GAA are required to achieve maximum uptake into myoblasts via CI-MPR compared to untagged GAA.

It has also been shown that the GILT tag does not interfere with the enzymatic activity of GAA.

### GAA PNP Assay

GAA enzyme was incubated in 50 μl reaction mixture containing 100 mM sodium acetate pH 4.2 and 10 mM Para-Nitrophenol (PNP) α-glucoside substrate (Sigma N1377). Reactions were incubated at 37° C. for 20 minutes and stopped with 300 μl of 100 mM sodium carbonate. Absorbance at 405 nm was measured in 96-well microtiter plates and compared to standard curves derived from p-nitrophenol (Sigma N7660). 1 GAA PNP unit is defined as 1 nmole PNP hydrolyzed/hour.

### GAA 4 MU Assay

GAA enzyme was incubated in 20 μl reaction mixtures containing 123 mM sodium acetate pH 4.0 with 10 mM 4-methylumbelliferyl α-D-glucosidase substrate (Sigma, catalog #M-9766). Reactions were incubated at 37° C. for 1 hour and stopped with 200 μl of buffer containing 267 mM sodium carbonate, 427 mM glycine, pH 10.7. Fluorescence was measured with 355 nm excitation and 460 nm filters in 96-well microtiter plates and compared to standard curves derived from 4-methylumbelliferone (Sigma, catalog #M1381). 1 GAA 4 MU unit is defined as 1 nmole 4-methylumbelliferone hydrolyzed/hour.

Specific activities of exemplary GILT-tagged GAA and wild-type, untagged GAA are shown in Table 2. The enzymatic activity of GILT-tagged GAA is comparable to an untagged GAA.

TABLE 2
Specific activity and Kₘ for GILT-tagged GAA ZC-701
and wild-type, untagged GAA.

| | ZC-701 | wtGAA |
|---|---|---|
| Specific Activity* | 315,000 | 346,000 |
| 4MU (nMoles/hr/mg) | | |
| Kₘ 4MU (mM)** | 1.47 | 1.41 |
| Kₘ PNP (mM)** | 3.29 | 9.21 |

*Average of determination for 3 preparations.
**Average of determination for 2 preparations

### Example 6

### Half-Life of GAA in Rat L6 Myoblasts

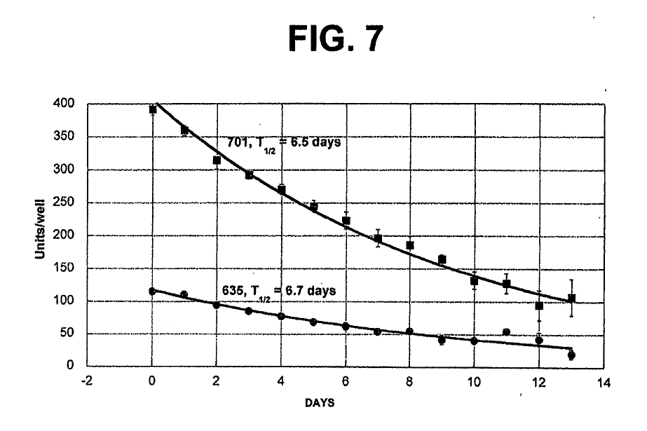

An uptake experiment was performed as described above (see Example 4) with GILT-tagged GAA and untagged GAA in rat L6 myoblasts. After 18 hours, media containing enzyme was aspirated off of cells and the cells were washed 4 times with PBS. At this time, duplicate wells were lysed (Time 0) and lysates were frozen at −80. Each day thereafter, duplicate wells were lysed and stored for analysis. After 14 days, all of the lysates were assayed for GAA activity. Data is plotted according to the 1^{st} order decay equation: ln Cₜ=−kt+ln C₀, where C is the concentration of compound, t is time in hours, and k is the 1^{st} order rate constant. FIG. 7 is an exemplary graph showing the half-life of GILT-tagged GAA ZC-701 and wild-type, untagged GAA (ZC-635) in rat L6 myoblasts. The results shown in FIG. 7 indicate that the tagged and untagged proteins have very similar half-lives, 6.5 and 6.7 days, respectively. This indicates that once inside cells, the GILT-tagged enzyme persists with similar kinetics to untagged GAA.

### Example 7

### Processing of GAA after Uptake

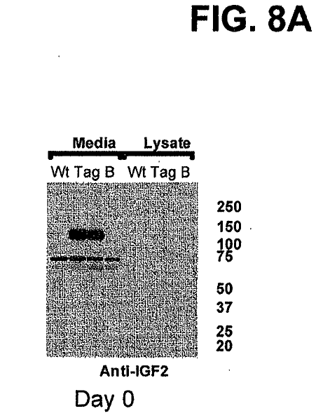

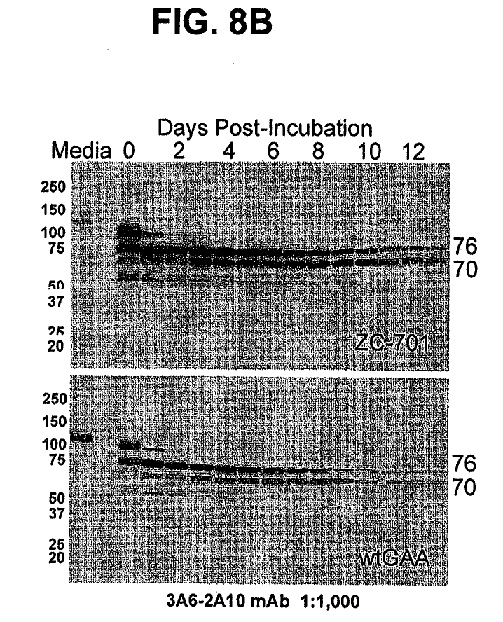

Mammalian GAA typically undergoes sequential proteolytic processing in the lysosome as described by Moreland et al. (2005) J. Biol. Chem., 280:6780-6791 and references contained therein. The processed protein gives rise to a pattern of peptides of 70 kDa, 20 kDa, 10 kDa and some smaller peptides. To determine whether the GILT-tagged GAA is processed similarly to the untagged GAA, aliquots of lysates from the above uptake experiment were analyzed by Western blot. FIGS. 8A-B are Western blots showing proteolytic processing of GILT-tagged GAA after uptake into rat L6 myoblasts. FIG. 8A is a Western blot showing the pattern of peptides identified by a monoclonal antibody that recognizes the 70 kDa IGF-II peptide and larger intermediates with the IGF-II tag. The results shown in FIG. 8A indicate loss of the GILT tag immediately after uptake. FIG. 8B is a Western blot showing processing of wild-type and GILT-tagged GAA into 76 kDa and 70 kDa species after uptake identified by a monoclonal antibody that recognizes the 70 kDa peptide and larger intermediates. The profile of polypeptides identified in this experiment was virtually identical for both the tagged and untagged enzyme. This indicates that once entering the cell, the GILT tag is lost and the GILT-tagged GAA is processed similarly to untagged GAA. Therefore it is likely that the GILT tag has little or no impact on the behavior of GAA once it is inside the cell.

### Example 8

### Pharmacokinetics

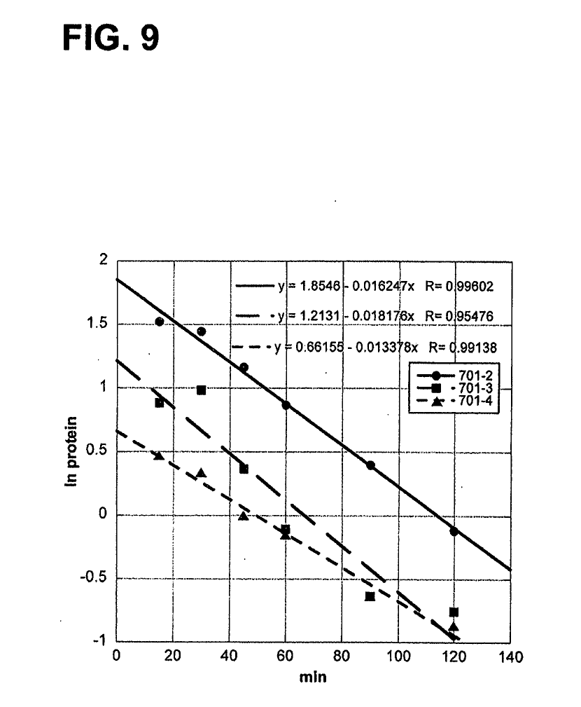

Pharmacokinetics of GILT-tagged GAA produced under different culture conditions was measured in wild-type 129 mice. GILT-tagged GAA ZC-701 was produced under 3 different culture conditions. Three groups of three 129 mice were injected in the jugular vein with a single dose of 10 mg/kg ZC-701 purified from culture supernatants of cells grown in 3 alternate media. Serum samples were taken preinjection and at 15 min, 30 min, 45 min, 60 min, 90 min, 120 min, 4 hours, and 8 hours post injection. The animals were then sacrificed. Serum samples were assayed by quantitative western blot. Data was plotted according to the 1^{st} order decay equation: ln Cₜ=−kt+ln C₀, where C is the concentration of compound, t is time in hours, and k is the 1^{st} order rate constant. Half-lives were obtained from the linear portion of the log plots which are illustrated in FIG. 9. The half-lives for the GILT-tagged GAA proteins were: Red line, PF-CHO, t½=43 min; Orange line, CDM4, t½=38 min; Green line, CD17, t½=52 min. Based on these results, the protein produced in CD 17 media has the most favorable half-life. These results indicate that the GILT-tagged GAA is not cleared from the circulation excessively rapidly.

### Example 9

### Tissue Half-Life of GAA

The objective of this experiment was to determine the rate at which GILT-tagged GAA activity is lost once the enzyme reaches its target tissue. In the Pompe mouse model, Myozyme® appears to have a tissue half-life of about 6-7 days in various muscle tissues (Application Number 125141/0 to the Center for Drug Evaluation and Research and Center for Biologics Evaluation and Research, Pharmacology Reviews).

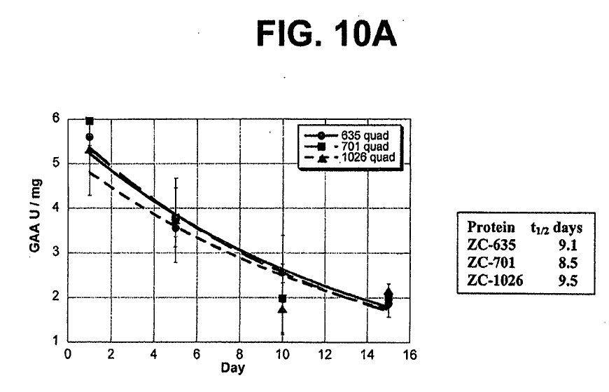

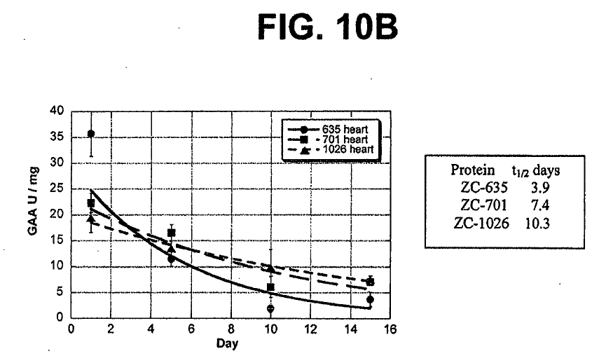

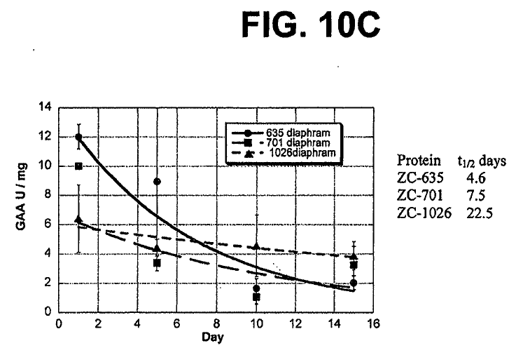

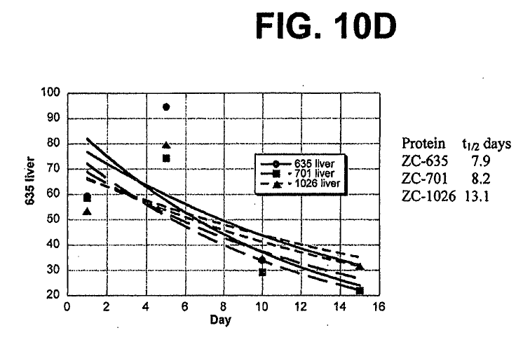

Pompe mice (Pompe mouse model 6^{neo}/6^{neo} as described in Raben (1998) JBC, 273:19086-19092, the disclosure of which is hereby incorporated by reference) were injected in the jugular vein with 10 mg/kg of either untagged GAA (ZC-635), or GILT-tagged GAA ZC-701, or GILT-tagged GAA ZC-1026. Mice were then sacrificed at 1, 5, 10, and 15 days post injection. Tissue samples were homogenized and GAA activity measured according to standard procedures. The tissue half-life of GILT-tagged GAA ZC-701 and ZC-1026 and the untagged GAA ZC-635 were calculated from the decay curves in different tissues (FIG. 10A, quadriceps tissue; FIG. 10B, heart tissue; FIG. 10C, diaphragm tissue; and FIG. 10D, liver tissue). The calculated half-life values are summarized in Table 3.

The tissue half-life for the untagged protein (ZC-635) ranged from 9.1 to 3.9 days in different tissues while the half-life for GILT-tagged GAA ZC-701 ranged from 8.5 to 7.4 in different tissues (Table 3). These ranges are likely to reflect statistical variation due to the relatively small sample size (3 animals per point) rather than significant differences.

For comparison, the half-lives in rat L6 myoblasts for ZC-701 and untagged wild-type GAA (ZC-635) calculated from the decay curves shown in FIG. 9 were 6.5 and 6.7 days, respectively.

TABLE 3
Tissue half-life of tagged and untagged GAA in various tissues.

| Tissue | ZC-701 T½, days | ZC-635 T½, days |
|---|---|---|
| Quad | 8.5 | 9.1 |
| Heart | 7.4 | 3.9 |
| Diaphragm | 7.5 | 4.6 |
| Liver | 8.2 | 7.9 |
| Rat L6 myoblasts | 6.5 | 6.5 |

These data indicate that once inside cells in Pompe mice, GILT-tagged GAA appears to persist with kinetics similar to the untagged GAA. Furthermore, the knowledge of the decay kinetics of the GILT-tagged GAA can help in the design of appropriate dosing intervals.

### Example 10

### Uptake of GILT-Tagged GAA into Lysosomes of C2C12 Mouse Myoblasts

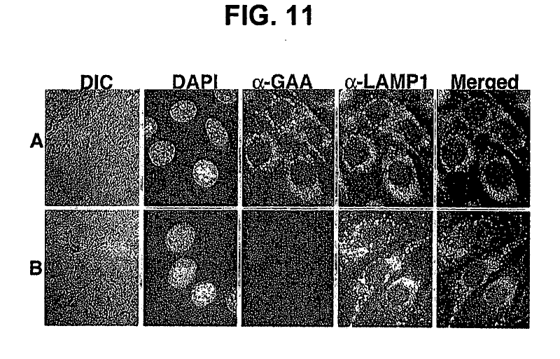

C2C12 mouse myoblasts were grown on poly-lysine coated slides (BD Biosciences) and incubated for 18 hours in the presence (Panel A) or absence (Panel B) of 100 nM GILT-tagged GAA at 37° C. in 5% CO₂. Cells were then incubated in growth media for 1 hour, then washed four times with D-PBS before fixing with methanol at room temperature for 15 minutes. The following incubations were all at room temperature, each separated by three washes in D-PBS. Incubations were for 1 hour unless noted. Slides were permeabilized with 0.1% triton X-100 for 15 minutes, then blocked with blocking buffer (10% heat-inactivated horse serum (Invitrogen) in D-PBS). Slides were incubated with primary mouse monoclonal anti-GAA antibody 3A6-1F2 (1:5,000 in blocking buffer), then with secondary rabbit anti-mouse IgG AF594 conjugated antibody (Invitrogen A11032, 1:200 in blocking buffer). A FITC-conjugated rat anti-mouse LAMP-1 (BD Pharmingen 553793, 1:50 in blocking buffer) was then incubated. Slides were mounted with DAPI-containing mounting solution (Invitrogen) and viewed with a Nikon Eclipse 80i microscope equipped with fluorescein isothiocyanate, texas red and DAPI filters (Chroma Technology). Images were captured with a photometric Cascade camera controlled by MetaMorph software (Universal Imaging). Images were merged using Photoshop software (Adobe). FIG. 11 shows the co-localization of signal detected by anti-GAA antibody with signal detected by antibody directed against a lysosomal marker, LAMP1. Therefore, this result demonstrates that GILT-tagged GAA is delivered to lysosomes.

### Example 11

### Clearance of Glycogen In Vivo

The objective of this experiment was to determine the rate at which glycogen is cleared from heart tissue after a single IV injection of GILT-tagged GAA or wt GAA into Pompe mice.

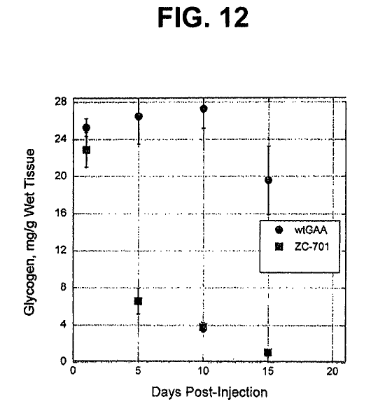

Pompe mice (Pompe mouse model 6^{neo}/6^{neo} as described in Raben (1998) JBC, 273:19086-19092, the disclosure of which is hereby incorporated by reference) were injected in the jugular vein with 10 mg/kg of either untagged GAA (ZC-635), or GILT-tagged GAA (ZC-701). Mice were then sacrificed at 1, 5, 10, and 15 days post injection. Each data point represents the average from three mice. Heart tissue samples were homogenized according to standard procedures and analyzed for glycogen content. Glycogen content in these tissue homogenates was measured using A. niger amyloglucosidase and the Amplex® Red Glucose assay kit (Invitrogen) essentially as described by Zhu et al. (2005) Biochem J., 389:619-628. Results displayed in FIG. 12 indicate that heart tissue from mice treated with ZC-701 showed almost complete clearance of glycogen whereas mice treated with the wt GAA showed only a small change in glycogen content.

### Example 12

### Reversal of Pompe Pathology

It has been shown that the therapeutic fusion protein of the present invention is therapeutically more effective than wt GAA in vivo. A study was conducted to compare the ability of ZC-701 and wt GAA to clear glycogen from skeletal muscle tissue in Pompe mice. Pompe mouse model 6^{neo}/6^{neo} animals were used (Raben (1998) JBC 273:19086-19092). Groups of Pompe mice (5/group) received four weekly IV injections of one of two doses of wt GAA or ZC-701 (5 mg/kg or 20 mg/kg) or vehicle. Five untreated animals were used as control, and received four weekly injection of saline solution. Animals received oral diphenhydromine, 5 mg/kg one hour prior to injections 2, 3, and 4. Pompe knockout mice in this study were injected with 25 μg of ZC-701 subcutaneously into the scruff of the neck when the animals were less than 48 hours old in order to tolerize them. Mice were sacrificed one week after the fourth injection, and tissues (diaphragm, heart, lung, liver, soleus, quadriceps, gastrocnemius, TA, EDL, tongue) were harvested for histological and biochemical analysis. Glycogen content in the tissue homogenates was measured using A. niger amyloglucosidase and the Amplex Red Glucose assay kit. The study design is summarized in Table 4.

TABLE 4

| | | | | Post-Injection | |
|---|---|---|---|---|---|
| Mice | Injection | Dose | | Survival Time | # Animals |
| GAA skin−/− | PBS | 0 | | 1 week after | N = 6 |
| | | | | final injection | |
| GAA skin−/− | Wt GAA | 5, 20 | mg/kg | 1 week after | N = 6/ |
| | (ZC-635) | | | final injection | dose |
| | | | | | Total = 12 |
| GAA skin−/− | GILT- | 5, 20 | mg/kg | 1 week after | N = 6/ |
| | modified | | | final injection | dose |
| | GAA | | | | Total = 12 |
| | (ZC-701/ | | | | |
| | ZC-1026) | | | | |

GAA enzyme levels in different tissue homogenates were measured using standard procedures and the results are summarized in Table 5.

TABLE 5
GAA levels in tissues.

| | | units/mg | |
|---|---|---|---|
| Tissues | GAAs | protein | SD |
| Gastrocnemius | 635-20 | 15.918 | 9.659 |
| | 701-20 | 7.495 | 1.435 |
| | 701-5 | 7.263 | 0.859 |
| | 635-5 | 4.828 | 0.251 |
| | PBS | 4.380 | 0.193 |
| Quadriceps | 635-20 | 9.164 | 3.297 |
| | 701-20 | 6.715 | 1.408 |
| | 701-5 | 6.158 | 1.140 |
| | 635-5 | 4.363 | 0.145 |
| | PBS | 4.018 | 0.298 |
| Diaphram | 635-20 | 42.178 | 53.517 |
| | 701-20 | 24.945 | 27.799 |
| | 701-5 | 7.795 | 0.387 |
| | 635-5 | 6.364 | 1.058 |
| | PBS | 5.254 | 0.281 |
| Heart | 635-20 | 5.121 | 2.082 |
| | 701-20 | 5.363 | 0.683 |
| | 701-5 | 6.330 | 1.310 |
| | 635-5 | 4.354 | 0.652 |
| | PBS | 3.911 | 0.311 |
| Tongue | 635-20 | 10.418 | 3.901 |
| | 701-20 | 5.668 | 0.568 |
| | 701-5 | 4.786 | 0.327 |
| | 635-5 | 4.783 | 0.494 |
| | PBS | 4.587 | 0.470 |
| Soleus | 635-20 | 34.397 | 22.049 |
| | 701-20 | 20.621 | 6.685 |
| | 701-5 | 13.618 | 2.804 |
| | 635-5 | 7.310 | 1.236 |
| | PBS | 7.490 | 3.137 |
| TA | 635-20 | 19.623 | 13.242 |
| | 701-20 | 7.148 | 1.318 |
| | 701-5 | 7.398 | 0.507 |
| | 635-5 | 4.688 | 0.140 |
| | PBS | 4.790 | 0.797 |
| EDL | 635-20 | 23.9828 | 12.6501 |
| | 701-20 | 13.0170 | 1.9414 |
| | 701-5 | 9.1218 | 0.7590 |
| | 635-5 | 7.2197 | 0.4682 |
| | PBS | 6.7099 | 1.1557 |

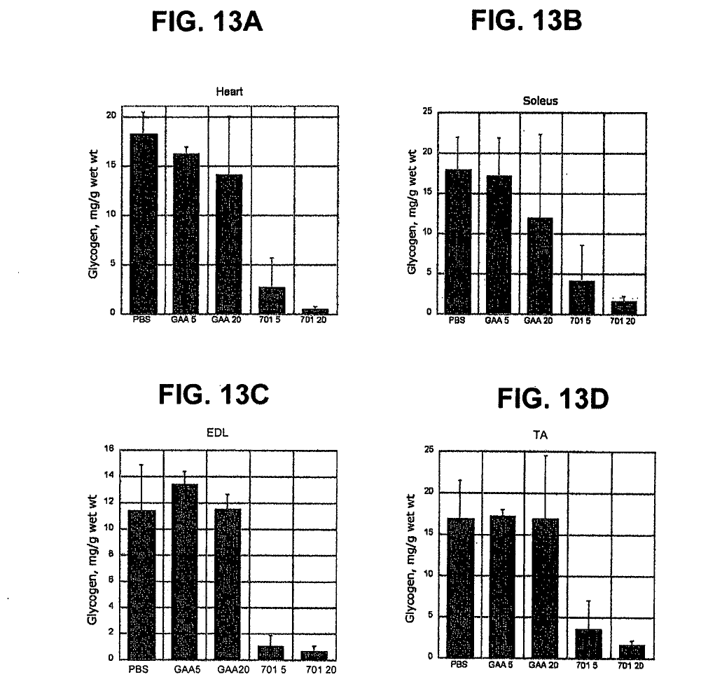

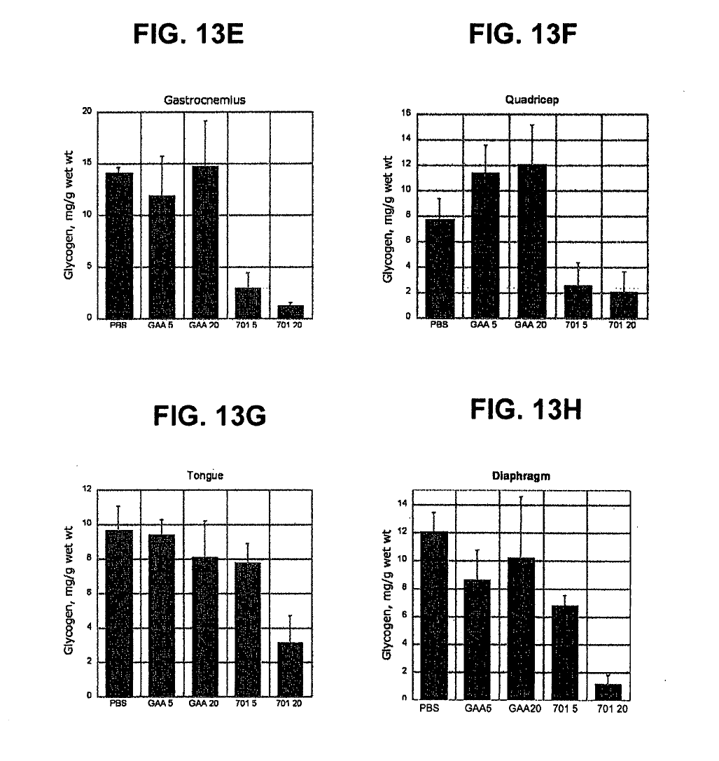

Glycogen content in these tissue homogenates was measured using A. niger amyloglucosidase and the Amplex® Red Glucose assay kit (Invitrogen) essentially as described by Zhu et al. (2005) Biochem J., 389:619-628. The glycogen data are depicted in FIGS. 13A-H. As used in FIGS. 13A-H, GAA 5 refers to untagged GAA at a dosage of 5 mg/kg; GAA 20 refers to untagged GAA at a dosage of 20 mg/kg; 701 5 refers to GILT-tagged GAA ZC-701 at a dosage of 5 mg/kg; 701 20 refers to GILT-tagged GAA ZC-701 at a dosage of 20 mg/kg. PBS was used as control. These results indicate the clear superiority of the GILT-tagged GAA (ZC-701) as compared to untagged GAA (ZC-635) in its ability to clear glycogen from a variety of muscle tissues. Specifically, ZC-701 was significantly more effective than wt GAA in its ability to clear glycogen from multiple skeletal muscle tissues. Pompe mice receiving wt GAA at either dose had glycogen levels that were not different from glycogen levels in PBS-treated animals. In contrast, animals receiving ZC-701 displayed significantly lower levels of glycogen.

### Example 13

### Optimization of Dosages Administration Intervals, and Age of Subjects

### Dosage

In the previous experiment, a dose of 5 mg/kg was almost as effective at clearing glycogen as was a dose of 20 mg/kg in a number of tissues. A dose titration experiment is used to determine the minimal effective dose that may be therapeutically sufficient in treating human patients. Five to seven tolerized Pompe knockout mice per group are injected weekly with different doses of GILT-tagged GAA. For example, tolerized Pompe mice are injected at 1.0, 1.5, 2, 2.5, 5, 10, 20 mg/kg.

The Pompe knockout mice are injected for 8 weeks and then sacrificed. Samples are taken from different tissues and the glycogen levels determined as described in Examples 11 and 12. Histochemistry for glycogen and enzyme distribution are also determined according to standard procedures. In addition, physiological measurements such as muscle force measurement and ventilation in response to hypercapnia using barometric whole-body plethysmography can be determined in the mice as described by Mah et al. (2007) Molecular Therapy (online publication).

### Intervals

In addition, treatment interval is evaluated and the maximum interval for a given dose that would still result in a clinical benefit is determined. Dose titrations are performed as described above with different treatment intervals, for example, injections every 2, 3, or 4 weeks. Glycogen clearance in skeletal muscle tissues such as, for example, soleus or quadriceps, is typically used as an indication to determine an optimal balance between dose and treatment interval in the mouse model. Other clinically relevant measurements as described above can be used as well.

For example, one experiment is designed to examine the effect of varied dosing intervals of GILT-tagged GAA on its efficacy in a Pompe mouse model. 2-3 month old Pompe mice (Raben JBC 1998 273:19086-19092) is divided into groups of 8 animals. One group receives weekly injections of PBS (the control group), one group receives weekly injections of 5 mg/kg GILT-tagged GAA, one group receives every other week an injection of 10 mg/kg GILT-tagged GAA, one group receives every third week an injection of 15 mg/kg GILT-taggd GAA, and one group receives an injection of 20 mg/kg GILT-tagged GAA every 4^{th} week. One week following week 12 injections, all animals are sacrificed. Tissue samples taken for analysis include: Heart, Soleus, Gastroc, EDL, TA, Quadricep, Psoas, Diaphragm, Brain, Tongue.

Analysis includes: biochemical glycogen analysis, histochemical stain for glycogen, EM on selected tissue, immunostaining on selected tissues, analysis of serum samples for antibody by ELISA, in vitro force-frequency measurement on Soleus muscle, glucose tetrasaccharide analysis in urine during in-life portion of study, 13C NMR spectroscopy determination of glycogen content pre and post treatment regimen.

A matrix of conditions in which dose and interval are varied can be generated to develop an understanding of the relationship between these parameters.

It is predicted that longer intervals between dosing at higher doses may prove effective thereby providing the rationale for a less burdensome treatment regimen for people suffering from Pompe disease. For example, based on the glycogen data shown in FIGS. 13A-H, it is predicted that weekly administration of a dose of 5 mg/kg would yield similar results to biweekly administration of 10 mg/kg or triweekly administration of 20 mg/kg. Therefore, it is expected that infusions of GILT-tagged GAA once every three or four weeks instead of once every two weeks will be sufficient o achieve therapeutic effects in human Pompe patients. Longer treatment intervals are highly advantageous because, at the least, it would reduce the burden and inconvenience on the patient.

### Age of Subjects

Effect of age of mice at initiation of therapy on therapeutic outcome is determined. It has been reported that, in Pompe mouse type II muscle fibers, autophagic vacuoles form over time which interfere with normal trafficking pathways including delivery of exogenous enzyme to lysosomes. See, Fukuda et al. (2006) Mol. Therapy, 14(6):831-839; Fukuda et al. (2006) Ann. Neurol., 59(4):700-708; Fukuda et al. (2006) Autophagy, 2(4):318-320. Scientists have shown that neo-rhGAA, which is recombinant GAA with up to 6 chemically coupled synthetic oligosaccharides containing 2 M6P each, can completely clear glycogen from 13 month old Pompe knockout mice. Zhu et al., (2005) Biochem J., 389:619-628. This suggests that the cellular pathology reported by Fukuda et al. may be reversible if one uses an enzyme with high affinity for the CI-MPR. Given the high affinity of GILT-tagged GAA for the CI-MPR and its more efficient delivery to muscle cells than untagged GAA, it is contemplated that sufficient GILT-tagged GAA enzyme can be delivered to lysosomes to clear glycogen and subsequently reverse the autophagic buildup. This is tested directly in 12-13 month old Pompe mice. These mice receive 4 weekly injections of 20 or 40 mg/kg GILT-tagged GAA and are sacrificed 1 week after the final injection. Glycogen content is assessed using the A. niger amyloglucosidase and the Amplex® Red Glucose assay kit (Invitrogen) essentially as described by Zhu et al. (2005). Glycogen is also assessed by histochemical staining as described by Lynch et al., (2005) J. Histochem. Cytochem., 53:63-73.

In addition, assays are performed to analyze the uptake of GILT-tagged GAA into isolated intact muscle fibers from animals with autophagic buildup to directly compare the ability of GILT-tagged GAA to target the lysosome under such conditions as described by Fukuda et al. as compared to untagged GAA. It is expected that the GILT-tagged GAA targets to the muscle fibers with autophagic buildup more efficiently than untagged enzyme.

### Experiment 14

### Human Clinical Studies

Based upon the success of animal treatments, a 6 month Phase I/II dose ranging study of GILT-tagged GAA in pediatric Pompe patients is designed. This clinical trial is an open-label, proof of concept human study performed to evaluate the safety, tolerability, efficacy, and pharmacokinetics of GILT-tagged GAA in patients with infantile-onset Pompe disease. In this study, the general treatment interval is once every two weeks (biweekly). An additional arm is expected to be added in which at a particular dose the treatment interval is once every 3 or 4 weeks.

A primary objective of the clinical trial includes determining the efficacy of 4 dose levels, namely 2.5, 5, 10, and 20 mg/kg, of GILT-tagged GAA administered by intravenous infusion every two weeks in treating patients with infantile-onset Pompe disease. Secondary objectives include (1) to evaluate the safety and pharmacokinetics of 4 different dose levels of GILT-tagged GAA administered by intravenous infusion every two weeks in treating patients with infantile-onset Pompe disease; (2) to determine the pharmacokinetics of 4 dose levels of GILT-GAA administered by intravenous infusion every two weeks in treating patients with infantile-onset Pompe disease; and (3) to determine the effect of each of the 4 dose levels of GILT-GAA administered by intravenous infusion every two weeks on the presence of muscle glycogen in patients with infantile-onset Pompe disease. A detailed protocol synopsis of this clinical trial is shown in Table 6.

TABLE 6
Human clinical trial.
PROTOCOL SYNOPSIS

| | |
|---|---|
| Title | An Open-Label, Proof of Concept Safety/Tolerability/Efficacy/PK Study of |
| | GILT-tagged Recombinant Human GAA in Patients with Infantile-Onset |
| | Pompe Disease |
| Objectives | Primary |
| | To determine the efficacy of 4 dose levels of Glycosylation- |
| | Independent Lysosomal Targeting (GILT)-tagged acid alpha glycosidase |
| | (GAA) administered by intravenous infusion every two weeks in treating |
| | patients with infantile-onset Pompe disease. |
| | Secondary |
| | To evaluate the safety of 4 dose levels of GILT-GAA administered |
| | by intravenous infusion every two weeks in treating patients with infantile- |
| | onset Pompe disease. |
| | To determine the pharmacokinetics of 4 dose levels of GILT-GAA |
| | administered by intravenous infusion every two weeks in treating patients |
| | with infantile-onset Pompe disease. |
| | Exploratory |
| | To determine the effect of each of the 4 dose levels of GILT-GAA |
| | administered by intravenous infusion every two weeks on the presence of |
| | muscle glycogen in patients with infantile-onset Pompe disease. |
| Study Design | This is a Phase I/open-label multiple dose study of 4 dose levels of GILT- |
| | GAA administered by intravenous infusion every two weeks in treating |
| | patients with infantile-onset Pompe disease. |
| | The study is comprised of a 2-week Screening period and a 26-week |
| | Treatment period. |
| Population | Main Inclusion Criteria: |
| | Patient's legal guardian(s) has provided written informed |
| | consent/authorization prior to any study-related procedures, |
| | Patient is male or female, 26 weeks of age or younger, and will be |
| | no older than 26 weeks of older when (s)he receives the first dose of GILT- |
| | GAA, |
| | Patient must have clinical symptoms (documented in his or her |
| | medical record) of infantile-onset Pompe disease, |
| | Patient must have endogenous GAA activity less than 1% of the |
| | normal range as assessed in cultured skin fibroblasts, |
| | Patient must have cardiomyopathy (left ventricular mass index |
| | >65 g/m²) by echocardiography, |
| | Patient's legal guardian(s) must ensure that the patient has the |
| | ability to comply with the protocol. |
| | Main Exclusion Criteria: |
| | Patient has symptoms of respiratory insufficiency including: |
| | an oxygen saturation <90% on room air as measured by pulse |
| | oximetry OR |
| | venous pCO2 >55 mmHg on room air or arterial pCO2 >40 mmHg |
| | on room air OR |
| | any ventilator use at the time of enrollment, |
| | Patient has a major congenital abnormality other than Pompe |
| | disease, |
| | Patient has clinically significant organic disease (with the exception |
| | of symptoms relating to Pompe disease), including clinically significant |
| | cardiovascular, hepatic, pulmonary, neurologic, or renal disease, or other |
| | medical condition, serious intercurrent illness, or extenuating circumstance |
| | that, in the opinion of the investigator, would preclude participation in the |
| | trial or potentially decrease survival, |
| | Patient has received any investigational medication within 30 days |
| | prior to the first dose of study drug or is scheduled to receive any |
| | investigational drug other than GILT-GAA during the course of the study, |
| | Patient has received enzyme replacement therapy with GAA from |
| | any source, |
| | Patient has previously been admitted to the study. |
| Planned Sample | 16 patients (4 patients per treatment group to receive one of four dose levels |
| Size | of GILT-GAA). |
| Number of Centers | The study will be conducted at approximately 8 sites across the US. |
| Study Drug | GILT-GAA for intravenous administration over 6 hours every two weeks |
| Formulation, | for 26 weeks (14 doses total). |
| Dosage & | |
| Administration | |
| Efficacy | LVMI by doppler echocardiography |
| Parameters | Alberta Infant Motor Scale (AIMS) |
| | Crying vital capacity |
| Safety Measures | Physical examination |
| | Weight |
| | Vital signs |
| | ECG |
| | Hematology, chemistry, and urinalysis laboratory tests |
| | Chest X-Ray |
| | Adverse events |
| | Antibodies to GILT-tagged GAA |
| Pharmacokinetics | Pharmacokinetics following dosing on Day 1, Week 12, and Week 26. |
| | Exact sampling times to be determined. |
| Pharmacodynamics | A muscle biopsy will be taken at Day 1, Week 12, and Week 26 to |
| | determine levels of muscle glycogen. |
| Statistical Methods | The sample size is consistent with sound clinical judgment and known and |
| | predicted pharmacology of GILT-GAA. Four patients per treatment group |
| | will provide adequate safety, efficacy, and pharmacokinetic, and |
| | pharmacodynamic data at each dose level. |

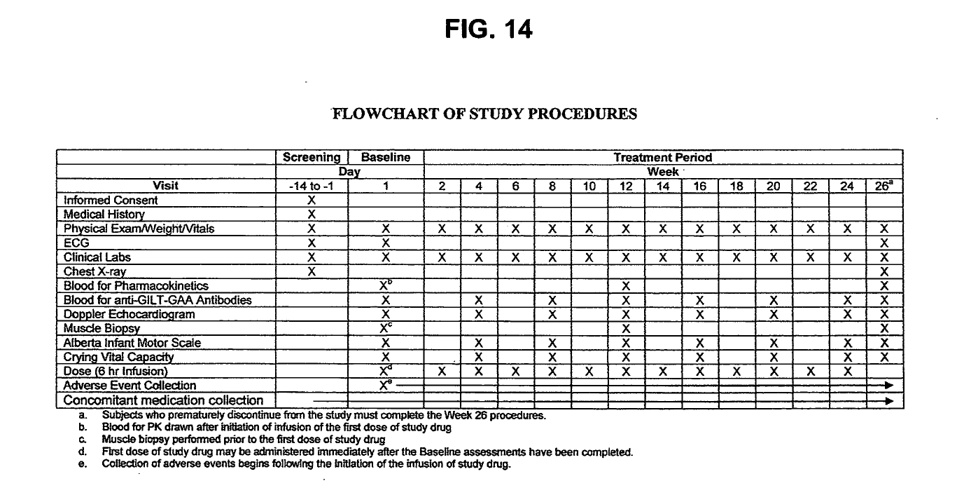

FIG. 14 shows a detailed flowchart of the clinical study procedures.

Additionally, it is desirable to include a step to tolerize or to immunologically suppress patients. In the clinical trials of other lysosomal enzyme replacement therapies, many patients were observed to produce high titers of antibodies against GAA. For example, this phenomenon has been observed with Gaucher patients taking Cerezyme®. In that case the majority of patients naturally became tolerized and stopped producing antibodies in response to the treatment regimen. Without wishing to be bound by theory, it is thought that the anti-GAA antibody will interfere with the targeting of the enzyme to the CI-MPR, thereby altering the biodistribution of the enzyme. One tolerizing strategy is to treat the Pompe patient with Rituximab®, a monoclonal antibody against CD20, before or during GILT-tagged GAA treatment. The dosage of Rituximab® used on Pompe patients is similar to that used in treating some autoimmune diseases as taught in Sperr et al. (2007) Haematologica, January; 92(1):66-71, the teachings of which are hereby incorporated by reference. This compound can also be used in conjunction with other immunosuppressive agents such as steroids.

The Pompe patients treated with GILT-tagged GAA are expected to demonstrate significant clearance of glycogen after 10-12 weeks based on histochemical staining of biopsy material. Thurberg et al. devised a classification of Pompe disease based on the continuum of ultrastructural damage into 5 stages of cellular pathology. Thurberg et al. (2006) Lab. Invest., 86:1208-1220. For example, early disease stage 1 cells contain small glycogen filled lysosomes between intact myofibrils. Stage 3 cells contain numerous glycogen filled lysosomes with much glycogen leaking into the cytoplasm due to the rupture of lysosomal membranes. These stages seem to correlate with the autophagic accumulations described by Fukada et al. Analysis of cellular pathology in the patients in the study at the outset of the treatment is expected to indicate the clinical outcomes. The responding patients generally have low percentage of myocytes with more severe forms of cellular pathology. In particular, patients with greater than 50% stage 2 myocytes have better clinical outcomes. It is also contemplated that younger patients generally have better clinical outcomes and patients having higher percentage of Type I muscle fibers also have better clinical outcomes.

Factors that modulate the severity of the cellular pathology include the presence of residual GAA activity in the patient due to the nature of the patient's GAA alleles, age of patient at the outset of treatment, and patient's immune response. For example, antibody response to GILT-tagged GAA is more severe in CRIM-negative patients.

Based on the results from initial animal experiments, GILT-tagged GAA is expected to be more effective than untagged GAA in treatment of human patients. It is expected that given a similar dose, a higher fraction of patients with a given level of cellular pathology at the outset of enzyme replacement therapy will respond favorably to the GILT-tagged GAA therapy. For example, in the pivotal clinical trial for Myozyme®, 12 of 18 patients had greater than 20% reduction in muscle glycogen at 52 weeks. However, only 3 of 18 patients experienced a 50% or greater decrease in glycogen content. Kishnani et al. (2007) Neurology, 68:99-109. Based on animal data and the work of Zhu et al., it is expected that GILT-tagged GAA will result in 80% reduction in glycogen content in most of the patients. A greater fraction of patients treated with GILT-tagged GAA is expected to show improvements in physiological parameters such as in motor function, respiration and more patients are expected to survive 1, 2, and 5 years after the onset of therapy.

Patients who start the enzyme replacement therapy with more advanced myocyte cellular pathology, for example, patients who are older than 6 months at the start of therapy, are also expected to have a significant reduction in muscle glycogen, improvements in respiratory capacity, motor function and better long term outcomes on GILT-tagged GAA enzyme replacement therapy.

### INCORPORATION OF REFERENCES

All publications and patent documents cited in this application are incorporated by reference in their entirety to the same extent as if the contents of each individual publication or patent document were incorporated herein.

1. A method for treating pompe disease in a subject comprising administering to the subject a therapeutically effective amount of a fusion protein comprising human acid alpha-glucosidase (GAA), or a fragment thereof, and a lysosomal targeting domain, wherein the lysosomal targeting domain binds the human cation-independent mannose-6-phosphate receptor in a mannose-6-phosphate-independent manner.

2. The method of claim 1, wherein the lysosomal targeting domain comprises mature human insulin-like growth factor II (IGF-II) or a fragment or sequence variant thereof.

3. The method of claim 2, wherein the lysosomal targeting domain comprises amino acids 1 and 8-67 of mature human IGF-II.

4. The method of claim 1, wherein the fusion protein comprises amino acids 70-952 of human GAA.

5. The method of claim 1, wherein the fusion protein has reduced mannose-6-phosphate (M6P) level thereon compared to wild-type human GAA.

6. The method of claim 1, wherein the fusion protein has no functional M6P level thereon.

7. The method of claim 1, wherein the therapeutically effective amount is in the range of 2.5-20 mg per kilogram of body weight of the subject.

8. The method of claim 1, wherein the fusion protein is administered intravenously.

9. The method of claim 1, wherein the fusion protein is administered bimonthly, monthly, triweekly, biweekly, weekly, daily, or at variable intervals.

10. A method for treating Pompe disease in a subject comprising administering to the subject a therapeutically effective amount of a fusion protein comprising amino acids 1 and 8-67 of mature human insulin-like growth factor II (IGF-II) and amino acids 70-952 of human acid alpha-glucosidase (GAA).

11. The method of claim 10, wherein the fusion protein further comprises a spacer sequence Gly-Ala-Pro between the amino acids of mature human IGF-II and the amino acids of human GAA.

12. The method of claim 10, wherein the fusion protein has reduced mannose-6-phosphate (M6P) level thereon compared to wild-type human GAA.

13. The method of claim 10, wherein the fusion protein has no functional M6P level thereon.

14. A method for reducing glycogen levels in vivo comprising administering to a subject suffering from Pompe disease an effective amount of a fusion protein comprising human acid alpha-glucosidase (GAA), or a fragment thereof, and a lysosomal targeting domain, wherein the lysosomal targeting domain binds the human cation-independent mannose-6-phosphate receptor in a mannose-6-phosphate-independent manner.

15. The method of claim 14, wherein the lysosomal targeting domain comprises mature human insulin-like growth factor II (IGF-II) or a fragment or sequence variant thereof.

16. The method of claim 15, wherein the lysosomal targeting domain comprises amino acids 1 and 8-67 of mature human IGF-II.

17. The method of claim 14, wherein the fusion protein comprises amino acids 70-952 of human GAA.

18. The method of claim 14, wherein the fusion protein has reduced mannose-6-phosphate (M6P) level thereon compared to wild-type human GAA.

19. The method of claim 14, wherein the fusion protein has no functional M6P level thereon.

20. The method of claim 14, wherein the therapeutically effective amount is in the range of 2.5-20 mg per kilogram of body weight of the subject.

21. The method of claim 14, wherein the fusion protein is administered intravenously.

22. The method of claim 14, wherein the fusion protein is administered bimonthly, monthly, triweekly, biweekly, weekly, daily, or at variable intervals.

23. A method for reducing glycogen levels in a mammalian lysosome comprising targeting to the lysosome an effective amount of a fusion protein comprising human acid alpha-glucosidase (GAA), or a fragment thereof, and a lysosomal targeting domain, wherein the lysosomal targeting domain binds the human cation-independent mannose-6-phosphate receptor in a mannose-6-phosphate-independent manner.

24. The method of claim 23, wherein the lysosomal targeting domain comprises mature human insulin-like growth factor II (IGF-II) or a fragment or sequence variant thereof.

25. The method of claim 23, wherein the lysosomal targeting domain comprises amino acids 1 and 8-67 of mature human IGF-II.

26. The method of claim 23, wherein the fusion protein comprises amino acids 70-952 of human GAA.

27. A method for reducing glycogen levels in a muscle tissue of a subject suffering from Pompe disease comprising delivering to the muscle tissue a therapeutically effective amount of a fusion protein comprising human acid alpha-glucosidase (GAA), or a fragment thereof, and a lysosomal targeting domain, wherein the lysosomal targeting domain binds the human cation-independent mannose-6-phosphate receptor in a mannose-6-phosphate-independent manner.

28. The method of claim 27, wherein the muscle tissue is skeletal muscle.

29. A method for treating cardiomyopathy associated with Pompe disease in a subject comprising administering to the subject a therapeutically effective amount of a fusion protein comprising human acid alpha-glucosidase (GAA), or a fragment thereof, and a lysosomal targeting domain, wherein the lysosomal targeting domain binds the human cation-independent mannose-6-phosphate receptor in a mannose-6-phosphate-independent manner.

30. A method for treating myopathy associated with Pompe disease in a subject comprising administering to the subject a therapeutically effective amount of a fusion protein comprising human acid alpha-glucosidase (GAA), or a fragment thereof, and a lysosomal targeting domain, wherein the lysosomal targeting domain binds the human cation-independent mannose-6-phosphate receptor in a mannose-6-phosphate-independent manner.

31. A method for increasing acid alpha-glucosidase (GAA) activity in a subject suffering from Pompe disease comprising administering to the subject a fusion protein comprising human acid alpha-glucosidase (GAA), or a fragment thereof, and a lysosomal targeting domain, wherein the lysosomal targeting domain binds the human cation-independent mannose-6-phosphate receptor in a mannose-6-phosphate-independent manner.

32. A pharmaceutical composition suitable for treatment of Pompe disease comprising a therapeutically effective amount of a fusion protein comprising human acid alpha-glucosidase (GAA), or a fragment thereof, and a lysosomal targeting domain, wherein the lysosomal targeting domain binds the human cation-independent mannose-6-phosphate receptor in a mannose-6-phosphate-independent manner.

33. The pharmaceutical composition of claim 32, wherein the lysosomal targeting domain comprises mature human insulin-like growth factor II (IGF-II) or a fragment or sequence variant thereof.

34. The pharmaceutical composition of claim 32, wherein the lysosomal targeting domain comprises amino acids 1 and 8-67 of mature human IGF-II.

35. The pharmaceutical composition of claim 32, wherein the fusion protein comprises amino acids 70-952 of human GAA.

36. The pharmaceutical composition of claim 32, wherein the fusion protein comprises amino acids 70-952 of human GAA and amino acids 1 and 8-67 of mature human IGF-II.

37. The pharmaceutical composition of claim 36, wherein the fusion protein further comprises a spacer sequence Gly-Ala-Pro between the amino acids of human GAA and the amino acids of mature human IGF-II.

38. The pharmaceutical composition of claim 32, wherein the fusion protein has reduced mannose-6-phosphate (M6P) level thereon compared to wild-type human GAA.

39. The pharmaceutical composition of claim 32, wherein the fusion protein has no functional M6P level thereon.

40. The pharmaceutical composition of claim 32, wherein the pharmaceutical composition further comprises a pharmaceutical carrier.
